(19) United States
(12) Patent Application Publication
(10) Pub. No.: US 2025/0000580 A1
(43) Pub. Date: Jan. 2, 2025

(54) SMART ASPIRATION SYSTEM AND LASER ACTIVATION SYSTEM

(71) Applicant: Eximo Medical LTD, Rehovot (IL)

(72) Inventors: Yoel ZABAR, Nes Ziona (IL); Ilan BEN OREN, Modiin (IL); Oren Meshulam Stern, Shilo (IL); Eitan Yaacov POLLACK, Efrat (IL)

(73) Assignee: Eximo Medical LTD, Rehovot (IL)

(21) Appl. No.: 18/884,248

(22) Filed: Sep. 13, 2024

Related U.S. Application Data
(63) Continuation-in-part of application No. 18/055,897, filed on Nov. 16, 2022.
(60) Provisional application No. 63/538,311, filed on Sep. 14, 2023.
(60) Provisional application No. 63/403,692, filed on Sep. 2, 2022.
(60) Provisional application No. 63/279,912, filed on Nov. 16, 2021.

(51) Int. Cl. A61B 18/24; A61B 18/00

(52) U.S. Cl. CPC A61B 18/245; A61B 2018/0041; A61B 2018/00648; A61B 2018/00666; A61B 2018/00708; A61B 2018/00744; A61B 2018/00827; A61B 2018/00863; A61B 2018/00904; A61B 2218/007

(57) ABSTRACT

Systems and methods for dynamically modulating aspiration and energy radiation in response to sensed conditions. An aspiration system can include a catheter configured to be inserted within a vasculature of the subject, a canister coupled to the catheter, a pressure source that generates a vacuum pressure through the catheter for aspirating the fluid, an energy source that generates energy radiation distally of the catheter, a sensor configured to sense a parameter associated with at least one of the catheter, the canister, or the pressure source, and a computer system coupled to the sensor. The computer can cause the pressure source to receive a measurement of the parameter from the sensor, determine whether the measurement violates a threshold associated with the parameter, and modulate the aspiration flow rate and/or energy radiation at the catheter tip in response to a determination that the measurement violates the threshold.

### PRIORITY

The present application claims priority to U.S. Provisional Patent Application No. 63/538,311, titled LASER ACTIVATION SYSTEM, filed Sep. 14, 2023. The present application is a continuation-in-part of U.S. Nonprovisional patent application Ser. No. 18/055,897, titled SMART ASPIRATION SYSTEM, filed Nov. 16, 2022, which claims priority to U.S. Provisional Patent Application No. 63/279,912, titled SMART ASPIRATION SYSTEM, filed Nov. 16, 2021, and U.S. Provisional Patent Application No. 63/403,692, titled SMART ASPIRATION SYSTEM, filed Sep. 2, 2022, each of which is hereby incorporated by reference herein in its entirety.

### BACKGROUND

Many of the most common and deadly diseases afflicting mankind result from or in the presence of undesirable material, most notably blood clots, in the blood vessels and heart chambers. Examples of such diseases include myocardial infarction, stroke, pulmonary embolism, deep venous thrombosis, atrial fibrillation, infective endocarditis, and so on. The treatment of some of these conditions, which involve smaller blood vessels, such as myocardial infarction and stroke, has been dramatically improved in recent years by targeted mechanical efforts to remove blood clots from the circulatory system. Other deadly conditions, which involve medium to large blood vessels or heart chambers, such as pulmonary embolism (½ million deaths per year) or deep venous thrombosis (2-3 million cases per year) have not benefited significantly from such an approach. Present treatment for such conditions with drugs or other interventions is not sufficiently effective. As a result, additional measures are needed to help save lives of patients suffering from these conditions.

In the systemic circulation, this undesirable material can cause harm by obstructing a systemic artery or vein. Obstructing a systemic artery interferes with the delivery of oxygen-rich blood to organs and tissues (arterial ischemia) and can ultimately lead to tissue death or infarction. Obstructing a systemic vein interferes with the drainage of oxygen-poor blood and fluid from organs and tissues (venous congestion) resulting in swelling (edema) and can occasionally lead to tissue infarction.

Many of the most common and deadly human diseases are caused by systemic arterial obstruction. The most common form of heart disease, such as myocardial infarction, results from thrombosis of a coronary artery following disruption of a cholesterol plaque. The most common causes of stroke include obstruction of a cerebral artery either from local thrombosis or thromboemboli, typically from the heart. Obstruction of the arteries to abdominal organs by thrombosis or thromboemboli can result in catastrophic organ injury, most commonly infarction of the small and large intestine. Obstruction of the arteries to the extremities by thrombosis or thromboemboli can result in gangrene.

In the systemic venous circulation, undesirable material can also cause serious harm. Blood clots can develop in the large veins of the legs and pelvis, a common condition known as deep venous thrombosis (DVT). DVT arises most commonly when there is a propensity for stagnated blood (long-haul air travel, immobility) and clotting (cancer, recent surgery, especially orthopedic surgery). DVT causes harm by (1) obstructing drainage of venous blood from the legs leading to swelling, ulcers, pain and infection and (2) serving as a reservoir for blood clot to travel to other parts of the body including the heart, lungs (pulmonary embolism) and across a opening between the chambers of the heart (patent foramen ovale) to the brain (stroke), abdominal organs or extremities.

In the pulmonary circulation, the undesirable material can cause harm by obstructing pulmonary arteries, a condition known as pulmonary embolism. If the obstruction is upstream, in the main or large branch pulmonary arteries, it can severely compromise total blood flow within the lungs and therefore the entire body, resulting in low blood pressure and shock. If the obstruction is downstream, in large to medium pulmonary artery branches, it can prevent a significant portion of the lung from participating in the exchange of gases to the blood resulting low blood oxygen and build up of blood carbon dioxide. If the obstruction is further downstream, it can cut off the blood flow to a smaller portion of the lung, resulting in death of lung tissue or pulmonary infarction.

Depending upon the state of the undesirable material, the undesirable material can be eliminated by mechanical means. Mechanical treatments involve the direct manipulation of the material to eliminate the obstruction. This can involve aspiration, maceration, and compression against the vessel wall, or other types of manipulation. The distinct advantage of mechanical treatment is that it directly attacks the offending material and eliminates the vascular obstruction independent of the specific content of the offending material. Mechanical treatments, if feasible, can usually prove to be superior to biologic treatments for vascular obstruction. Procedural success rates tend to be higher. The best example of this advantage is in the treatment of acute myocardial infarction. Although thrombolytic therapy has had a major impact on the management of patient with myocardial infarction, this option is now relegated to a distant second choice. The clear standard of care today for an acute myocardial infarction is an emergency percutaneous coronary intervention during which the coronary artery obstruction is relieved by aspiration, maceration, or balloon compression of the offending thrombus. This mechanical approach has been shown to decrease the amount of damaged heart tissue and improve survival relative to the thrombolytic biological approach.

Catheter pulmonary embolectomy, where the pulmonary emboli are removed percutaneously using one of several techniques, can be subdivided into three categories. With fragmentation thrombectomy, the clot is broken into smaller pieces, most of which migrate further downstream, decreasing the central obstruction but resulting in a “no-reflow” phenomenon. It is sometimes used in combination with thrombolytics which preclude their use as an alternative to thrombolytics. With the rheolytic thrombectomy, high velocity saline jets create a Venturi effect and draw the fragments of the clot into the catheter. Finally, the aspiration techniques draw the clot into a catheter via suction. With a Greenfield embolectomy, the catheter with the attached clot is repeatedly drawn out of the vein. All of these techniques rely on catheters which are small compared to the size of the clots and blood vessels. Their limited success is likely related to their inability to achieve a complete en bloc removal of the material without fragmentation.

Some currently existing systems utilized for clearing vascular debris are designed to aspirate the subject's blood to assist in capturing and removing the vascular debris. However, there are several problems with such systems when aspirating blood, including removing or aspirating a large volume of blood and the aspiration lumen becoming blocked or occluded during a procedure (which, in turn, limits aspiration efficiency). If the vacuum level of the aspiration system becomes compromised, it can result in an incomplete removal of the undesirable material and increase the risk of emboli. In addition, applying high aspiration rates at the catheter tip may induce injury to the vessel if the high aspiration rates are applied without a blockage being present.

Accordingly, there is a need in the prior art for vascular treatment systems utilizing aspiration that are able to control the aspiration flow rate in a smart, efficient manner in response to changing conditions during treatment of the subject.

### SUMMARY

The present disclosure is directed to control systems for vascular treatment systems that are configured to aspirate the subject's blood during removal of the undesirable intravascular material.

In one embodiment, the present disclosure is directed to an aspiration system comprising: a catheter configured to be inserted within a vasculature of the subject; a canister coupled to the catheter, the canister configured to receive fluid from the catheter; a pressure source coupled to the catheter, the pressure source configured to generate a vacuum pressure through the catheter for aspirating the fluid; a sensor configured to sense a parameter associated with at least one of the catheter, the canister, or the pressure source; and a computer system coupled to the sensor, the computer system comprising a processor and a memory, the memory storing instructions that, when executed by the processor, cause the computer system to: cause the pressure source to initiate the vacuum pressure throughout the catheter, receive a measurement of the parameter from the sensor, determine whether the measurement violates a threshold associated with the parameter, and modulate the aspiration flow rate in response to a determination that the measurement violates the threshold.

In one embodiment, the present disclosure is directed to a computer-implemented method for removing undesirable intravascular material (UIM) from a subject using a system, the system comprising a catheter configured to be inserted within a vasculature of the subject, a canister coupled to the catheter, the canister configured to receive fluid and the UIM from the catheter, a pressure source coupled to the catheter, the pressure source configured to generate a vacuum pressure through the catheter for aspirating the fluid and the UIM, and a sensor configured to sense a parameter associated with at least one of the catheter, the canister, or the pressure source, the method comprising: causing, by a computer system coupled to the pressure source and the sensor, the pressure source to initiate the vacuum pressure throughout the catheter; receiving, by the computer system, a measurement of the parameter from the sensor, determining, by the computer system, whether the measurement violates a threshold associated with the parameter; and modulating, by the computer system, the aspiration flow rate in response to a determination that the measurement violates the threshold.

In one embodiment, the present disclosure is directed to a system for aspiration of fluid from the body comprising: an aspiration catheter; a waste container coupled to the aspiration catheter, the waste container configured to receive the aspirated fluid from the body; a pump coupled to the catheter, the pump configured to generate a negative pressure through the catheter; a weight sensor configured to sense a parameter associated with the waste container; a pressure sensor configured to sense the negative pressure; and a computer system coupled to the sensor, the computer system comprising a processor and a memory, the memory storing instructions that, when executed by the processor, cause the computer system to: cause the pump to initiate the negative pressure, receive a first measurement of the parameter from the weight sensor and a second measurement of the negative pressure from the pressure sensor, determine whether at least one of the first measurement or the second measurement violates a threshold associated with the parameter or the negative pressure, and modulate the aspiration flow rate in response to a determination that at least one of the first measurement or the second measurement violates the threshold.

In some embodiments, the system can further comprise a sensor configured to detect a UIM within the system.

In some embodiments, the UIM comprises a soft thrombus.

In some embodiment, the sensor configured to detect the UIM comprises at least one of an optical sensor, an ultrasonic sensor, an inductive sensor, a magnetic sensor, a sensor configured to detect electric conductivity, or a turbine sensor.

In some embodiments, the system can further comprise a filter configured to capture a UIM within the system.

In some embodiments, the filter is positioned between the canister and the catheter.

In some embodiments, the filter is positioned within the canister.

In some embodiments, the sensor is configured to determine a weight of the filter and the computer system is configured to subtract the weight of the filter from a weight of the canister to determine one or more parameters associated with the system.

In one embodiment, the present disclosure is directed to a system for removing UIM from a vasculature of a subject. The system may comprise a catheter configured to be inserted within the vasculature of the subject; a canister coupled to the catheter, the canister configured to receive fluid and UIM from the catheter; a pressure source coupled to the catheter, the pressure source configured to generate a vacuum pressure through the catheter for aspirating the fluid and the UIM; an energy source coupled to the catheter, the energy source configured to generate energy radiation distally of the catheter to the UIM; a sensor configured to sense a parameter associated with at least one of the catheter, the canister, or the pressure source; and a computer system coupled to the sensor, the computer system comprising a processor and a memory, the memory storing instructions that, when executed by the processor, cause the computer system to: cause the pressure source to initiate the vacuum pressure throughout the catheter, receive a measurement of the parameter from the sensor, determine whether the measurement violates a threshold associated with the parameter, and modulate the aspiration flow rate and/or the energy radiation at a tip of the catheter in response to a determination that the measurement violates the threshold.

In one embodiment, the present disclosure is directed to a computer-implemented method for removing UIM from a subject using a system. The system may comprise a catheter configured to be inserted within a vasculature of the subject, a canister coupled to the catheter, the canister configured to receive fluid and the UIM from the catheter, a pressure source coupled to the catheter, the pressure source configured to generate a vacuum pressure through the catheter for aspirating the fluid and the UIM, an energy source coupled to the catheter, the energy source configured to generate energy radiation distally of the catheter to the UIM; and a sensor configured to sense a parameter associated with at least one of the catheter, the canister, or the pressure source, the method comprising: causing, by a computer system coupled to the pressure source, the energy source and the sensor, the pressure source to initiate the vacuum pressure throughout the catheter; receiving, by the computer system, a measurement of the parameter from the sensor, determining, by the computer system, whether the measurement violates a threshold associated with the parameter; and modulating, by the computer system, the aspiration flow rate and/or the energy source at a tip of the catheter in response to a determination that the measurement violates the threshold.

In some embodiments, wherein the vacuum pressure is initiated to generate a first aspiration flow rate and the modulated aspiration flow rate is at a second aspiration flow rate, greater than the first aspiration flow rate.

In some embodiments, the modulating of the aspiration flow rate and/or energy radiation includes: modulating the aspiration flow rate at the tip of the catheter; receiving another measurement of the parameter from the sensor; determining whether the another measurement violates the threshold associated with the parameter; modulating the energy radiation at the tip of the catheter in response to a determination that the another measurement violates the threshold.

In some embodiments, the causing of the pressure source to initiate the vacuum pressure throughout the catheter includes initiating the vacuum pressure while the energy source is not activated.

In some embodiments, the modulating of the energy radiation includes activating the energy source to generate energy radiation distally of the catheter to the UIM.

In some embodiments, the determination that the measurement violates the threshold corresponds to the tip of the catheter being proximate the UIM in the vasculature or a clog present in the system.

In some embodiments, the threshold comprises a target aspiration flow rate; and modulation of the aspiration flow rate and/or energy radiation causes an aspiration flow rate of the fluid and the UIM to align with the target aspiration flow rate.

In some embodiments, the sensor is selected from the group consisting of a pressure sensor configured to sense the vacuum pressure, a weight sensor configured to sense a weight of the canister, a flow sensor configured to sense an amount of the aspiration, and a current sensor configured to sense a current drawn by the pressure source.

In one embodiment, the present disclosure is related to a method. The method may comprise: activating a pump to initiate a first vacuum pressure to be generated through the catheter to aspirate a fluid and UIM; receiving a first measurement of a parameter from the sensor; determining whether the first measurement violates a threshold associated with the parameter thereby determining a state of the system; wherein, when the first measurement does not violate the threshold, the first aspiration flow rate is maintained through the catheter, wherein, when the first measurement is determined to violate the threshold according to a first condition, a second aspiration flow rate is generated through the catheter, wherein the second aspiration flow rate is a higher aspiration flow rate than the first aspiration flow rate; wherein, when the first measurement is determined to violate the threshold according to a second condition, the energy source is activated to generate an energy radiation at the distal end of the catheter.

In some embodiments, when the first measurement does not violate the threshold, the state of the system is determined to be in a free flow state.

In some embodiments, when the measurement is determined to violate the threshold according to the first condition, the state of the system is determined to be in a UIM detected state.

In some embodiments, when the measurement is determined to violate the threshold according to the second condition, the state of the system is determined to be in obstructed state.

In some embodiments, when the measurement is determined to violate the threshold according to the second condition, the second aspiration flow rate is generated through the catheter while the energy source is activated.

In some embodiments, the method further comprises: receiving a second measurement of the parameter from the sensor; determining whether the second measurement violates the threshold associated with the parameter; wherein, when the second measurement does not violate the threshold, the energy source remains activated and the second aspiration flow rate is maintained through the catheter, wherein, when the second measurement is determined to violate the threshold according to a third condition, the energy source is deactivated and the aspiration flow rate is returned to the first aspiration flow rate through the catheter.

In some embodiments, the method further comprises modulating a control element to cause the aspiration flow rate within the catheter to be returned to the first aspiration flow rate.

In some embodiments, the method further comprises: modulating a control element to generate the second aspiration flow rate, wherein the control element includes: is selected from the group consisting of a valve configured to control the aspiration through tubing connecting the canister to the catheter, an air leak control element configured to modulate the vacuum pressure, a secondary pump configured to control the aspiration through the tubing, and a booster reservoir configured to modulate the vacuum pressure.

### BRIEF DESCRIPTION OF THE DRAWINGS

FIG. 1A shows a perspective view of a tip section of an illustrative hybrid catheter in accordance with an embodiment of the present disclosure.

FIG. 1B shows an end view of the tip section of the hybrid catheter of FIG. 1A.

FIG. 1C shows a cross-sectional view of the tip section of the hybrid catheter of FIG. 1A inside a vessel with partial plaque blockage.

FIG. 2 shows a view of the tip section of the hybrid catheter of FIG. 1A inside a vessel with partial plaque blockage.

FIG. 3A shows an image of an illustrative vascular treatment system in accordance with an embodiment of the present disclosure.

FIG. 3B shows a block diagram of the vascular treatment system of FIG. 3A.

FIG. 4A shows a block diagram of an illustrative vascular treatment system including a flow sensor in accordance with an embodiment of the present disclosure.

FIG. 4B shows a perspective view of the embodiment of the vascular treatment system shown in FIG. 4A.

FIG. 5A shows a block diagram of an illustrative vascular treatment system including a pressure sensor.

FIG. 5B shows a perspective view of the embodiment of the vascular treatment system shown in FIG. 5A.

FIG. 5C shows a block diagram of an illustrative vascular treatment system including a differential pressure sensor.

FIG. 6A shows a block diagram of an illustrative vascular treatment system including a pressure sensor assembly in accordance with an embodiment of the present disclosure.

FIG. 6B shows a perspective view of the embodiment of the vascular treatment system shown in FIG. 6A.

FIG. 7A shows a block diagram of an illustrative vascular treatment system including a weight sensor in accordance with an embodiment of the present disclosure.

FIG. 7B shows a perspective view of the embodiment of the vascular treatment system shown in FIG. 7A.

FIG. 8A shows a block diagram of an illustrative vascular treatment system including an air flow sensor in accordance with an embodiment of the present disclosure.

FIG. 8B shows a perspective view of the embodiment of the vascular treatment system shown in FIG. 8A.

FIG. 9 shows a block diagram of an illustrative vascular treatment system including a UIM sensor in accordance with an embodiment of the present disclosure.

FIG. 10A shows a block diagram of an illustrative vascular treatment system including a plurality of sensor types in accordance with an embodiment of the present disclosure.

FIG. 10B shows a block diagram of an illustrative vascular treatment system including a pressure sensor and a weight sensor in accordance with an embodiment of the present disclosure.

FIG. 10C shows a block diagram of an illustrative vascular treatment system including a UIM filter in accordance with an embodiment of the present disclosure

FIG. 11A shows a block diagram of an illustrative vascular treatment system including a valve control element in accordance with an embodiment of the present disclosure.

FIG. 11B shows a perspective view of a first embodiment of the vascular treatment system shown in FIG. 11A.

FIG. 11C shows a perspective view of a second embodiment of the vascular treatment system shown in FIG. 11A.

FIG. 12A shows a block diagram of an illustrative vascular treatment system including an air leak control element in accordance with an embodiment of the present disclosure.

FIG. 12B shows a perspective view of an embodiment of the vascular treatment system shown in FIG. 12A.

FIG. 13A shows a block diagram of an illustrative vascular treatment system including a second pump control element in accordance with an embodiment of the present disclosure.

FIG. 13B shows a perspective view of a first embodiment of the vascular treatment system shown in FIG. 13A.

FIG. 13C shows a perspective view of a second embodiment of the vascular treatment system shown in FIG. 13A.

FIG. 14 shows a block diagram of an illustrative vascular treatment system including a pressure source controller in accordance with an embodiment of the present disclosure.

FIG. 15 shows a block diagram of an illustrative vascular treatment system including a boost reservoir control element in accordance with an embodiment of the present disclosure.

FIG. 16 depicts a flow diagram for an illustrative process for modulating aspiration flow in a vascular treatment system based on a sensed parameter in accordance with an embodiment of the present disclosure.

FIG. 17 depicts a flow diagram for an illustrative process for modulating aspiration flow in a vascular treatment system based on whether an obstruction is identified in accordance with an embodiment of the present disclosure.

FIG. 18 depicts a flow diagram for an illustrative process for modulating aspiration flow in a vascular treatment system based on a target aspiration flow rate in accordance with an embodiment of the present disclosure.

FIG. 19 shows a block diagram of an illustrative vascular treatment system including an additional control element in accordance with an embodiment of the present disclosure.

FIG. 20 depicts a flow diagram for an illustrative process for modulating aspiration flow in a vascular treatment system based on whether a UIM is detected or a vessel wall is detected.

FIG. 21 shows a block diagram of an illustrative vascular treatment system including an energy source in accordance with an embodiment of the present disclosure.

FIG. 22 depicts a flow diagram for an illustrative process for modulating aspiration flow and/or energy radiation in a vascular treatment system based on a sensed parameter in accordance with an embodiment of the present disclosure.

FIG. 23 depicts a flow diagram for an illustrative process for modulating aspiration flow and/or energy radiation in a vascular treatment system based on whether an obstruction is identified in accordance with an embodiment of the present disclosure.

FIG. 24 depicts a flow diagram for an illustrative process for modulating aspiration flow and/or energy radiation in a vascular treatment system based on a target aspiration flow rate in accordance with an embodiment of the present disclosure.

FIG. 25 depicts a flow diagram for an illustrative process for modulating aspiration flow and/or energy radiation in a vascular treatment system based on whether a UIM is detected, a UIM has been aspirated or an obstruction is identified in accordance with an embodiment of the present disclosure.

FIG. 26 depicts a flow diagram for another illustrative process for modulating aspiration flow and/or energy radiation in a vascular treatment system based on whether a UIM is detected, a UIM has been aspirated or an obstruction is identified in accordance with an embodiment of the present disclosure.

FIG. 27 depicts a flow diagram for an illustrative process for modulating aspiration flow and/or energy radiation in a vascular treatment system based on whether a measurement violates a threshold in accordance with an embodiment of the present disclosure.

### DETAILED DESCRIPTION

This disclosure is not limited to the particular systems, devices and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “device” is a reference to one or more devices and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

As used herein, the term “undesirable intravascular material” (UIM) refers to intravascular debris including, but not limited to, blockages in a vessel due to plaque, calcium, atheroma, thrombus, embolus, clot, vegetative growth, infected vegetative growth (such as endocarditis), pulmonary embolism, tumor, arterial clots, undesirable material trapped in dialysis grafts and/or stents, and other undesirable natural and/or unnatural foreign bodies to be removed from a subject's body.

As used herein, the term “en bloc” refers to entirely, wholly, and/or without significant fragmentation.

As used herein, the terms “vacuum pressure,” “suction force,” and/or “vacuum force” refer to the negative pressure created by removing air from a space creating a pressure differential resulting in the force that a vacuum exerts upon the UIM. A drive force refers to the pressure differential generated by the device that exerts a force upon the UIM.

As used herein, the term “differential pressure” refers to the difference in pressure between two given points. Positive pressure refers to a pressure at a first point that is greater than a pressure at a second point. Negative pressure refers to a pressure at a first point that is lower than a pressure at a second point.

As used herein, the term “vacuum” refers to a differential pressure, including decreases in pressure (i.e., negative pressure) below atmospheric pressure.

As used herein, the term “aspiration flow rate” refers to the flow rate of aspirated fluid, blood, UIM, and/or other substances from the vasculature of the patient, through the vascular treatment system, in response to the vacuum pressure generated by a pressure source (e.g., a pump) and/or modulation of the control element. As used herein, “aspiration flow rate” relates at least in part to the aspiration force or negative pressure generated by modulation of the control element, pressure source, and/or combinations of the various elements of the treatment system. For example, modulating the aspiration flow rate to increase aspiration when a UIM and/or clog is detected at the tip of the catheter may include modulating the control element to increase the aspiration force at the tip of the catheter, but the actual flow rate of the UIM and blood through the vascular treatment system may temporarily be zero even though the system is in a high aspiration mode until the UIM is unclogged due to the application of the increased aspiration force. Conversely, modulating the aspiration flow rate to decrease aspiration when a UIM has been aspiration and/or clog has been cleared may include modulating the control element to decrease the aspiration force at the tip of the catheter thereby reducing the flow rate of any UIM and blood through the vascular treatment system.

Target vessels, treatment sites, or target areas include, but are not limited to, systemic venous circulation (e.g., inferior vena cava and/or superior vena cava, pelvic veins, leg veins, neck and arm veins); arterial circulation (e.g., aorta or its large and medium branches); heart chambers, such as in the left heart (e.g., the left ventricular apex and left atrial appendage), in the right heart (e.g., right atrium and right ventricle), or on its valves; small blood vessels; medium blood vessels; large blood vessels; iliofemoral vein; peripheral vasculature; and/or the pulmonary circulation (e.g., pulmonary veins and/or pulmonary arteries). In some embodiments, other treatment sites or target areas could include other nonvascular tubular structures, such as ducts or any other avascular tubular tissue. In some embodiments, other treatment sites or target areas could include pacemaker leads, stents, or other artificial implanted medical devices.

This disclosure relates to devices and methods for minimally invasive removal of UIM from a vessel or other hollow anatomical structure of a subject. In particular, the disclosure is directed to smart aspiration and laser activation control for atherectomy and thrombectomy systems.

### Vascular Treatment Systems

Described herein are systems and methods to facilitate the removal of UIM from the interior walls of a target vessel or treatment site of a subject. Although primarily described in the context of atherectomies, the embodiments described herein may be useful in various vascular applications, such as atherectomy, angioplasty, debulking of plaque in in-stent restenosis, leads extraction, thrombectomy in chronic peripheral and coronary artery diseases and for management of acute blockage of vessels in coronary and neurovascular applications and venous thrombectomy applications. Another example is the use of embodiments in gastroenterology, such as for removal of sessile and flat lesions in the GI tract, Barrett's Esophagus management and in analogous applications requiring removal of tissue from the inner walls in gynecology and urology interventions.

The embodiments described herein can make use of a “hybrid” catheter that utilizes a combination of laser and mechanical removal (also “debulking”) of UIM from a bodily lumen. In vascular interventions, the catheter may be configured to weaken and/or even cut and detach UIM with a laser and then, even in cases where the plaque material was not entirely removed, detaching the rest of the plaque material by mechanical means, such as using a blade. The laser may change the mechanical characteristics of tissue, and thereby improve performance of mechanical tools such as various types of blades or shavers. By way of example, the laser may make a soft tissue crispier so it can be effectively crushed using the mechanical tool.

According to some embodiments, the catheter comprises a tip section, which may be essentially in a cylindrical shape, having circumferentially directed laser optics, optionally in the form of one or more optical fibers, configured to deliver laser radiation, and a circular-action cutter including one or more blades configured to assist in cutting and/or detaching undesired materials (also “deposits”) from an inner surface of a blood vessel. The one or more optical fibers may be circumferentially directed, namely, they may be located along an inner surface of the cylindrical tip section, which is near the periphery of the tip section. Alternatively, the circumferentially directed optical fibers may be located elsewhere but directed, by way of orientation and/or optical focusing, to radiate an area in front of the circumference of the tip section.

The laser may be selected according to the selected resonator optics; for example, fluoride fiber lasers may be used to emit laser radiation on the 2.9 μm transition and Thulium fiber lasers may be used to emit radiation on the 1.9-2.1 μm transition. An advantage of an embodiment using a laser in the region of 2.9-3 micron is that the absorption is very high and results in a very short length of absorption, on the order of 15 microns. Therefore, the relaxation time is shorter so the pulse rate may be increased above 100 Hz in order to accelerate the procedure. In some embodiments, a 355 nm laser could be used because the energy from 355 nm lasers is highly absorbed in blood products and the laser energy can be delivered with standard fused silica fibers.

In addition to the laser beam that interacts with the undesired material, a laser with controlled pulse rate and/or power may be used to interact with the liquid between the fiber tip (exit of laser beam) and tissue, either to allow for “opening” of a passage for the beam (e.g., a channel where light is not absorbed when UV radiation is used) to the tissue prior and adjunctive to the required interaction with the tissue, and/or to facilitate the process (when mid-IR radiation is used) benefiting from the “water spray” effect. By way of clarification, the tip can be in mechanical contact with the tissue being ablated or not.

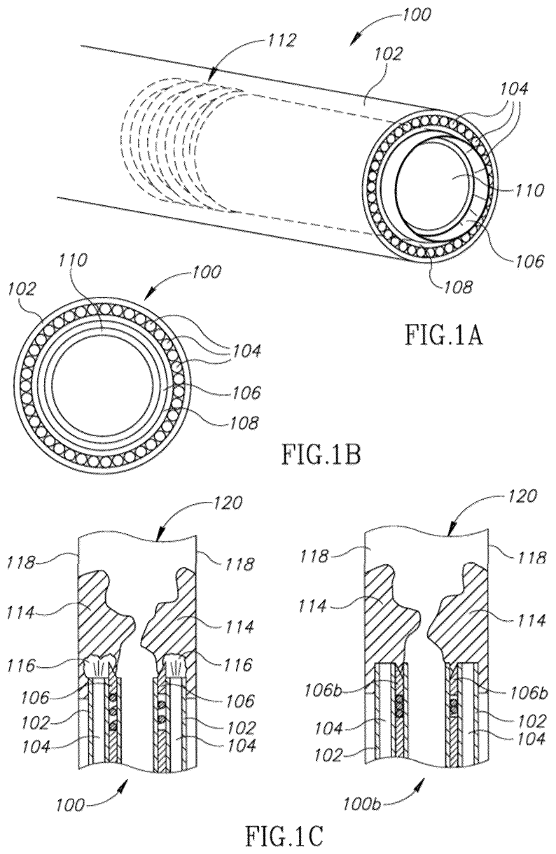

Reference is now made to FIGS. 1A, 1B and 1C, which show an exemplary cylindrical tip section 100 of a hybrid catheter in perspective, front and cross-section views, respectively, in accordance with an exemplary embodiment. The remainder of the catheter's shaft (not shown) may, in some embodiments, be biocompatible polymer tubing, optionally coated, to minimize friction with the vessel's walls.

Tip section 100 is positioned at the distal end (i.e., the end which is inserted into the blood vessel) of the hybrid catheter. Tip section 100 may include a housing 102, for example a cylindrical one, at least one optic fiber(s) 104 positioned along an inner surface of housing 102, and a circular-action cutter (or simply “cutter”) 106 positioned inwardly of the optic fibers. Alternatively, in an embodiment (not shown), the circular-action cutter may be positioned outwardly of the optic fibers. It is intended that the following description of the embodiments in which the circular-action cutter is positioned inwardly, be applied, mutatis mutandis, to the alternative, not-shown embodiment. Optionally, optic fiber(s) 104 are delimited and/or supported by a first inner wall 108. Further optionally, cutter 106 is delimited and/or supported by a second inner wall 110.

In accordance with some embodiments, the catheter is used with a standard guidewire.

In accordance with some embodiments, the catheter is connected to a suction pump that generates low pressure to collect undesired material, saline and/or the like through the catheter. The pump may be a peristaltic pump, which mounts externally to the fluid path, to avoid any contamination of the pump. Optionally, this obviates the need to use disposable parts. In other embodiments, a diaphragm pump or piston pump may be used.

Optic fibers 104, serving as the laser optics of the present hybrid catheter, may be connected, at their proximal end (not shown), to a laser source characterized by one or more of the parameters laid out herein. Optic fibers 104 may deliver the laser beams from the source towards the intervention site in the body. In tip section 100 of FIG. 1C, optic fibers 104 are shown as they emit laser towards undesired material 114. One or more areas 116 in undesired material 114 may consequently be modified or even ablated by the laser. Then, cutter 106 may more readily cut into undesired material 114 and detach at least a part of it from the vessel's walls 118.

Cutter 106 is optionally an annular blade extending to a certain depth inside tip section 100 and coupled to a suitable motor (not shown), located in the tip section or further in the shaft, supplying rotary and/or vibratory power to the blade. Optionally, one or more flexible members, such as a spring 112 (FIG. 1A), may interact with cutter 106 at its base, to allow it to retract and protrude from housing 102. Tip section 100 of FIGS. 1A-C is shown with cutter 106 in its protruding position, while tip section 100b of FIG. 1C is shown with the cutter, now marked 106b, in its retracted position. The length of protrusion of catheter 106 out of housing 102 may be, for example, up to about 350 microns when treating blood vessels. When protruding, cutter 106 is used for detaching undesired material (also “deposit”) 114 from an inner surface 118 of a blood vessel 120. According to some embodiments, when a certain force (for example, above a predetermined value) is applied to cutter 106 from the front, which may be a result of blockage in blood vessel 120, the cutter 106 may shift its position and retract into housing 102.

The annular blade of cutter 106 may have sufficiently thin edges, such as around 100 microns. Suitable blades may be tailor-made by companies such as MDC Doctor Blades, Crescent and UKAM. The blade may optionally be mounted at the end of a rotatable tube. Such tubes are available from manufacturers such as Pilling, offering a line of laser instrumentation and blade manufacture. The blade may be metal or manufactured by molding a material such as plastic, which is optionally coated with a coating having proper characteristics for in-vivo use. In other embodiments, the cutter 106 may include blunt blades at distal ends of the inner and outer walls of the catheter that house the optical fibers therebetween. Further, one or more portions of the catheter can be coated to aid in removal of the UIM. For example, coatings may be applied to the inner walls of the catheter to facilitate easy travel of the UIM through the catheter lumen. In further embodiments, coatings may be applied to the outer walls of the catheter to aid in pushability and maneuverability of the catheter through the vasculature.

An exemplary tip section may have an external diameter of approximately 3-5 mm, an internal diameter (within the innermost layer, be it the cutter or an extra wall) of approximately 2-4 mm, and optical fibers each having an approximately 0.07-0.2 mm diameter.

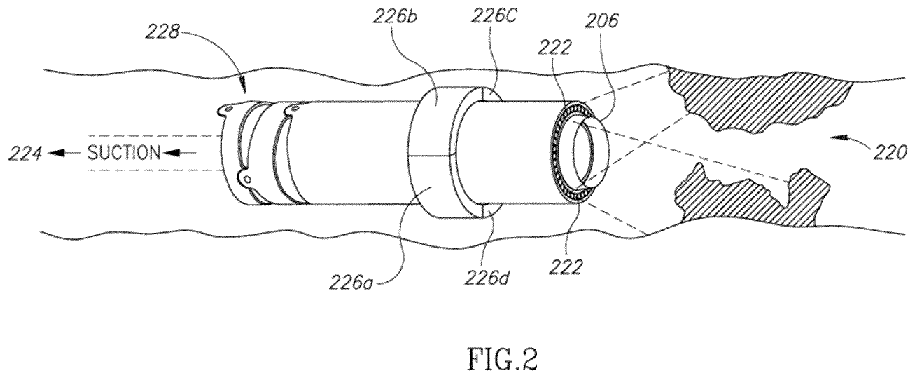

Reference is now made to FIG. 2, which shows an exemplary tip section 200 of a hybrid catheter, which may be similar to tip section 100 of FIG. 1 with one or more alterations: First, one or more fibers 222 of the optical fibers existing in tip section 200 may be used for imaging the lumen of a blood vessel 220 by transporting reflected and scattered light from inside the lumen to an external viewing and/or analysis device (not shown) located externally to the body. This may aid in avoiding perforation of vessel 220 and allowing for on-line monitoring of the intervention process. Second, tip section 200 may be maneuverable, so as to allow different viewing angles and/or in order to align the laser beams and a cutter 206 differently. Third, a cleaning channel (not shown) may be present inside tip section 200 and extending outside the body, through which channel suction 224 is applied in order to evacuate debris of the undesired material which were treated by the lasers and/or cutter 206. These optional alternations are now discussed in greater detail.

A conventional manner for detection of plaque and other lesions and for monitoring of vessel treatment is based on ultrasound and fluoroscopy. Here, however, one or more fibers 222 may be utilized for detection of lesions and/or to monitor the intervention process on-line, based on the reflection and/or scattering of the laser light from the vessel and/or the deposits. Alternatively or additionally, a different source of illumination may be used, such as through one or more other fibers. The captured light may be transmitted to a sensor such as a charge-coupled device (CCD), a metal-oxide-semiconductor (MOS), or a complementary MOS (CMOS). The sensing may include a filter or means for spectral imaging to gain information about the material characteristics (plaque, tissue, calcified plaque, blood clot, etc.). This may enable a quick and effective procedure with minimal risk of perforation and may enable debulking procedures wherein a guidewire cannot or should not be used.

The angle of tip section 200 may be controlled to enable, by means of tip deflection, material removal in a cross-section larger than the catheter size. This may be done by mechanical means, such as by selective inflation and deflation of at least two balloons (not shown) attached to the tip section externally at different angles, or a balloon with different compartments 226a-d. In another embodiment, the angle of the tip section 200 may be controlled by using links forming a joint 228. In such an embodiment, the links of the joint 228 may be controllable from outside the body using one or more wires (not shown).

The laser optics of some embodiments will now be discussed in greater detail. The laser beam may be directed through fibers each having a core diameter optionally in the range of 40-250 microns. In a configuration where the catheter's circumference is, for example, 15 mm, using fibers with an outer diameter of 50 microns will result in using approximately 300 fibers with a cross-section area smaller than 1 mm², so that for a coupling efficiency of 75%, the energy at the exit of each fiber will be close to 40 mJ/mm² when pumped with a 50 mJ laser. Adequate fibers for some embodiments may be all-silica fibers with a pure silica core. These fibers can usually withstand about 5 J/cm² in the input. Some embodiments include fibers with a numerical aperture (NA) in the range of 0.12-0.22. Examples of a relevant fiber are FiberTech Optica's SUV100/110AN fiber for UV application and the low OH version SIR100/140AN for use with a laser in the 1900-2100 nm range, and Infrared Fiber Systems, IR Photonics and A.R.T. Photonics GmbH fibers for transmission of radiation in the 2900-3000 nm range. Some embodiments may include microlenses at the tip area to manipulate the beam at the exit of each individual fiber.

The power required for effective ablation with 355 nm, 10 nsec pulses (approximately 30-60 mJ/mm²) is close to the damage threshold of certain fibers or above it, which may lead, in existing products, to the need of extended pulse length, for example. According to some embodiments, high peak power is maintained and, accordingly, the catheter may include means for delivery of the laser power through relatively bigger optical fibers, e.g., 100 or even 300 micron fibers that do not extend all the way to the end of the tip section.

Additional information regarding embodiments of atherectomy devices and/or systems can be found in U.S. patent application Ser. No. 16/436,650, published as U.S. Patent Application Pub. No. 2019/0321103A1, titled HYBRID CATHETER FOR VASCULAR INTERVENTION, filed Jun. 10, 2019; and U.S. patent application Ser. No. 17/395,799, published as U.S. Patent Application Pub. No. 2021/0361355A1, titled SYSTEM FOR TISSUE ABLATION USING PULSED LASER, filed Aug. 6, 2021, each of which is hereby incorporated by reference herein in its entirety.

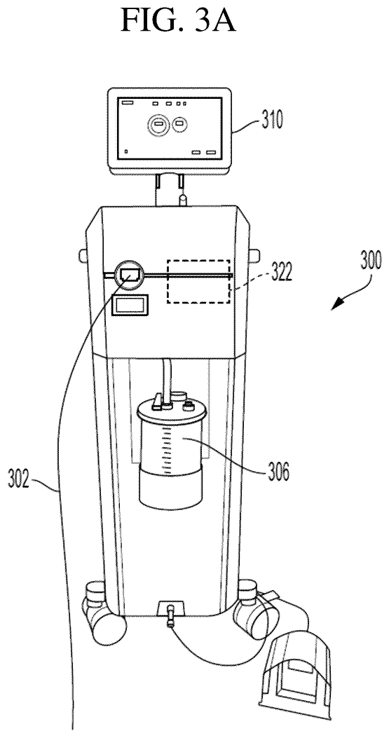

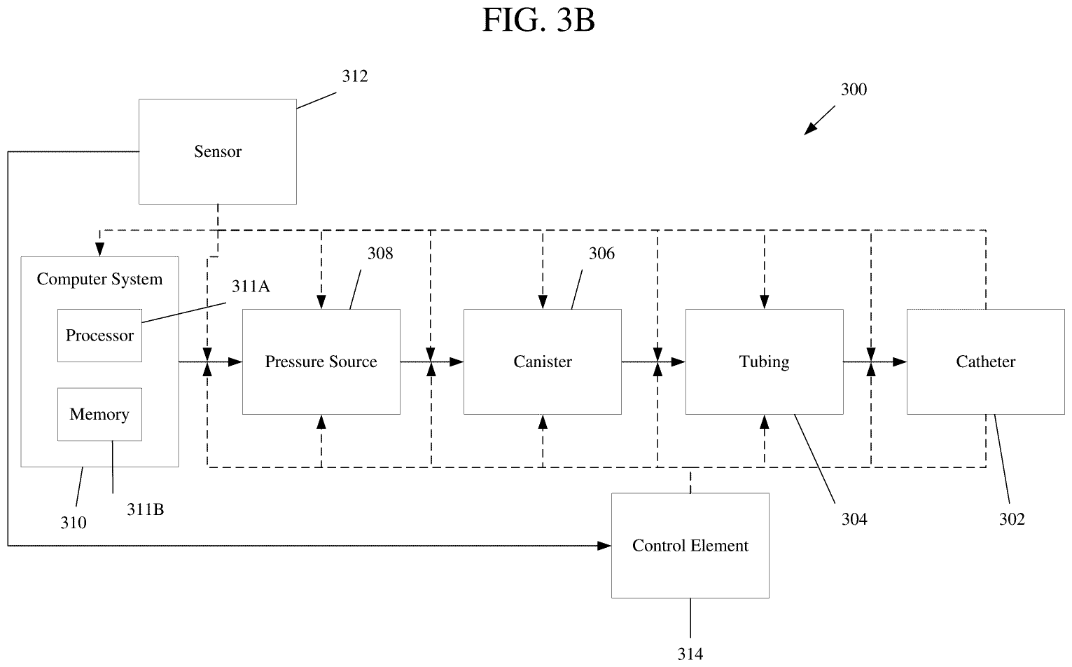

The hybrid catheter embodiments described herein can be used as components of a vascular treatment system 300, such as is illustrated in FIG. 3A and shown schematically in FIG. 3B. The vascular treatment system 300 can include a catheter 302 that is configured to be placed into the vasculature of a subject for the treatment and/or removal of UIM, such as the embodiments that are described above in connection with FIGS. 1A-2. The vascular treatment system 300 can further include a canister 306 (or “waste container”) coupled to the catheter 302 via tubing 304. The canister 306 can receive and store the blood and/or UIM that is removed from the subject during treatment for subsequent disposal. The catheter 302 can further be operably coupled to a pressure source 308 (e.g., a pump) that is configured to generate the suction 224 through the catheter 302 for removal and aspiration of the UIM. Further, the vascular treatment system 300 can include a computer system 310 for monitoring the status of the treatment procedure and controlling the operation of the other components of the system 300. The computer system 310 can include a processor 311A coupled to a memory 311B such that the processor 311A can execute instructions stored in the memory 311B to perform various functions embodied by the instructions. In some embodiments, the computer system 310 can provide a graphical user interface (GUI) that allows users to input various parameters, including a target aspiration flow rate, vacuum pressure levels, and so on. Accordingly, the vascular treatment system 300 can be utilized to aspirate UIM in either native or stented vasculature for removal of the UIM from the subject.

The aspiration flow rate results from the vacuum pressure generated by the pressure source 308. In other words, the pressure source 308 (e.g., a pump) generates a vacuum pressure (i.e., negative pressure) that draws fluid (e.g., blood) and/or UIM from the patient's vasculature, through the catheter 302 into the canister 306. Further, the vascular treatment system 300 can modulate the aspiration flow rate through the action of one or more control elements 314 through a variety of different mechanisms of action that are described in greater detail below. The control elements can change the aspiration flow rate by, for example, changing the vacuum pressure sensed by the catheter tip. In some embodiments, modulation of the control element(s) 314 increases or decreases an aspiration force or negative pressure at the distal tip of the catheter, thereby modulating the flow rate of UIM and/or blood aspirated into the system 300. The control element 314 can in turn be controlled by the computer system 310. In some embodiments, the computer system 310 can control the control element 314 to modulate the aspiration flow rate in response to measurements from one or more sensors 312, which is described in greater detail below.

In some embodiments, it can be advantageous to maintain a continuous aspiration flow rate during a procedure. In one embodiment, the vascular treatment system 300 could be configured to maintain an aspiration flow rate of 20 ml/min to 100 mL/min. In one embodiment, the vascular treatment system 300 could be configured to maintain an aspiration flow rate of 20 mL/min to 50 mL/min. In some embodiments, the pressure source 308 can be configured to generate from, for example and without limitation, 20 torr to 300 torr. If there is a blockage in the catheter 302 and/or other components of the system 300, the vacuum level can be increased (e.g., to about 25 torr to 100 torr) to clear the blockage. Under normal flow conditions (e.g., about 20 mL/min to about 50 mL/min), the vacuum level can be maintained at about 120 torr to 750 torr to minimize blood aspiration. In some embodiments, the generated aspiration flow rate may be modulated (i.e., increased or decreased), but never fully ceases. It can be advantageous to never fully cease the aspiration flow during treatment because blood coagulates when it stops flowing. Additionally, stopping flow entirely could risk clot particles being released from the catheter to the blood stream (i.e., falling back into the blood stream), thereby causing an emboly. Maintaining continuous flow overall throughout the treatment mitigates this risk. If the system 300 paused the aspiration flow for any reason, the subject's blood could coagulate, which could create additional blockages within the catheter 302 and/or other components of the system 300. Conversely, it would not be desirable for the system 300 to always be run at the highest vacuum pressure levels in order to attempt to avoid the formation of blockages because it would result in too much blood being removed from the subject. Accordingly, the ability of the vascular treatment system 300 to dynamically modulate the vacuum pressure sensed by the catheter tip can be advantageous because it allows the system to clear obstructions, while reducing the amount of blood loss in doing so. Therefore, it would be desirable for the vascular treatment system 300 to be able to dynamically shift between different vacuum levels based on sensed conditions within the system 300.

### Smart Aspiration for Vascular Treatment Systems

Vascular treatment systems, such as the embodiments described above, can be configured to aspirate the bodily fluids during removal of the targeted UIM. In some embodiments, the vascular treatment systems can include one or more sensors that are configured to sense various parameters associated with the system and modulate the vacuum level and/or aspiration flow rate at the tip of the catheter 302 using various control elements. FIG. 3B illustrates a diagram of a vascular treatment system 300 that includes a sensor 312 that is configured to sense one or more parameters associated with one or more components of the system 300 (or the connections between the components of the system 300) and a control element 314 that is configured to modulate the vacuum level and/or aspiration flow rate at catheter tip in response to the sensed parameter. In particular, the control element 314 can modulate the vacuum level and/or aspiration flow rate through a variety of different mechanisms of action (e.g., valves, booster reservoirs, or controlled leaks), which are described in greater detail below. Further, various embodiments of the vascular treatment system 300 can include a plurality of sensors 312 and/or a plurality of control elements 314. In one embodiment, the vascular treatment system 300 can modulate (i.e., increase or decrease) the vacuum level and/or aspiration flow rate via the action of the control element 314 without stopping the flow (i.e., causing the flow rate to be zero) during aspiration. This embodiment can be advantageous because when blood flow stops, the blood can begin to coagulate, which in turn can cause blockages (e.g., in the catheter 302) that can negatively impact the performance of the vascular treatment system 300. Another advantage of not stopping flow is that clot detection increases since flow is continuous and constantly being sensed by sensors 314 and not intermittently. Further, constant flow without stopping flow can mitigate risk of emboli.

As indicated in FIG. 3B, the sensor 312 can be configured to sense a parameter associated with the computer system 310, the pressure source 308, the canister 306, the tubing 304, the catheter 302, the control element 314, or any of the connections between the aforementioned components. As likewise indicated in FIG. 3B, the control element 314 can be operably coupled to the computer system 310, the pressure source 308, the canister 306, the tubing 304, the catheter 302, or any of the connections between the aforementioned components such that the control element 314 can control the corresponding component to modulate the vacuum level and/or aspiration flow rate for the vascular treatment system 300. In some embodiments, the sensor 312 and/or computer system 310 could further be coupled to the control element 314 and configured to sense a parameter and/or state of the control element 314. For example, in an embodiment where the control element 314 includes a valve, the valve could include an encoder or another device that is configured to output the state (e.g., position) of the valve to the computer system 310. Accordingly, the computer system 310 could sense a parameter and/or state of the control element 314 as part of the feedback control of the control element 314.

In various embodiments, the sensor 312 can include a pressure sensor, an air flow sensor, a pump current sensor, a level sensor, a weight sensor, a blood flow sensor, an ultrasound sensor, an optical sensor or a temperature sensor. In various embodiments, the control element 314 can include a solenoid valve, a pinch valve, a proportional pinch valve, a peristaltic pump, a pump controller (e.g., a pulse width modulation controller or a voltage controller), a pump with multiple pump heads, multiple pumps or a booster pump. Embodiments including various combinations of the sensors 312 and control elements 314 will be discussed in greater detail below. Additionally, in some embodiments, the sensor 312 can monitor the state of the control element 314. For example, the sensor 312 could monitor whether the control element 314 is opened or closed, or an amount that the control element 314 is opened or closed. In other embodiments, the control element 314 itself can include an internal sensor or encoder for monitoring openness or closedness of the control element 314.

As an example, when a normal or steady aspiration flow rate is observed within the system 300, such flow rate may be about 20-50 mL/min. At this state, the vacuum level at the catheter tip is maintained and held at about 120-750 torr. When the sensed aspiration flow rate is below about 20 mL/min, there may be a partial blockage (i.e., not a complete blockage or clog). In this state, the control element 314 could be utilized to increase the vacuum level as will be sensed by the catheter tip thereby helping to clear the partial blockage. When the sensed aspiration flow rate is below approximately 10 mL/min, there may be a complete blockage or clog. In this state, the control element 314 may be modulated to increase the vacuum level as will be sensed by the catheter tip (e.g., to 25-100 torr or to the maximum available vacuum level) thereby helping to clear the complete blockage or clog. In some embodiments, the computer system 310 may also be equipped with an alert for alerting the user that the computer system 310 is nearing a maximum level of blood aspiration by the system 300 (e.g., about 400 mL). In some embodiments, a first alert or warning can be triggered to notify a user of about 300 mL of blood aspiration by the system 300 and then once blood aspiration reaches about 400 mL, the computer system 310 can issue a second alert or warning or even be configured to automatically shut off.

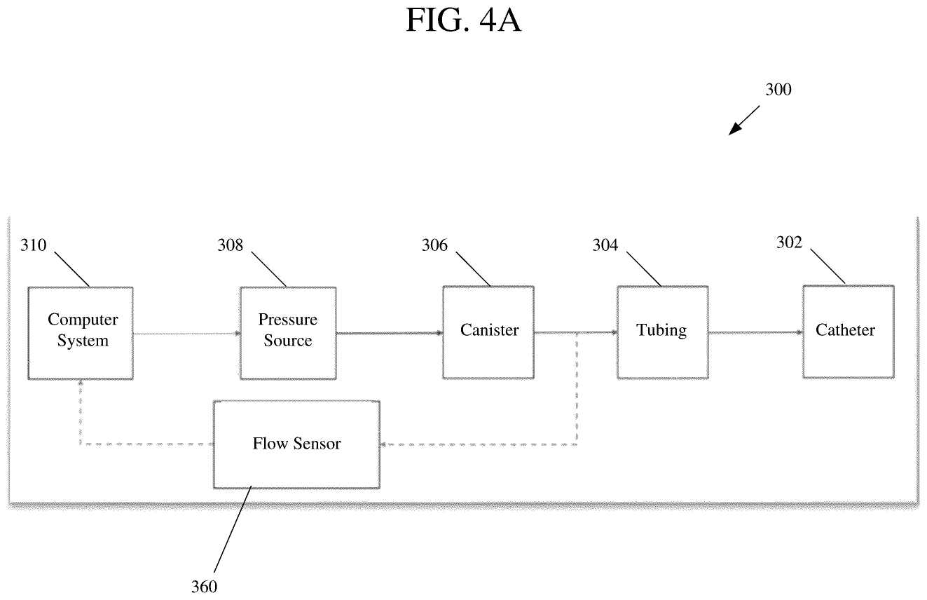

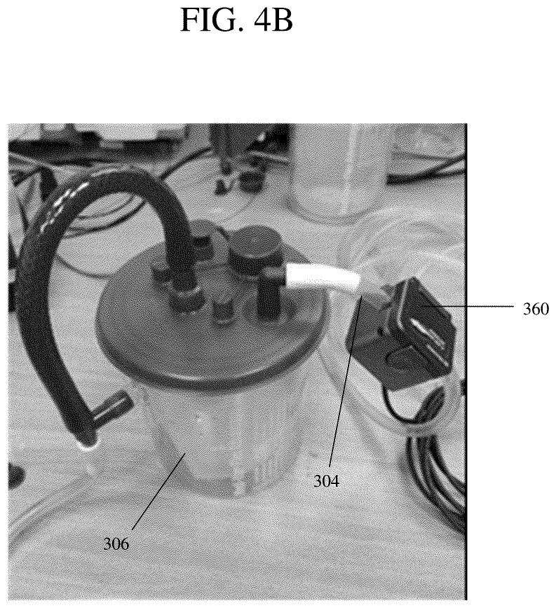

FIGS. 4A and 4B illustrate an embodiment of a vascular treatment system 300 where the sensor 312 includes a flow sensor 360. The flow sensor 360 could be positioned at different locations along or within the vascular treatment system 300. In one embodiment, the flow sensor 360 could be operably coupled to or along the tubing 304 coupling the canister 306 to the catheter 302. The flow sensor 360 can be configured to sense the rate of blood flow through the tubing 304. The flow sensor 360 can include a blood flow sensor, a temperature sensor, or a combination thereof. In an illustrative embodiment shown in FIG. 4B, the flow sensor 360 could include a contactless ultrasonic flow meter that is connected to one of the two ends of the tubing 304. The ultrasonic flow meter could be, for example, clamped over the tubing 304 and sense the flow therethrough. The ultrasonic flow meter can output a signal indicative of the sensed flow rate through the fluid line 330. In another illustrative embodiment (not shown), the flow sensor 360 could include a turbine infrared (IR) flow meter that is positioned in-line with the tubing 304. This flow meter can include a turbine component that is rotated as fluid flows therethrough and an IR sensor that can count the number of rotations of the turbine, which can in turn be used to determine the flow rate. Where a temperature sensor is used, the temperature sensor measures a temperature of the catheter 302 and/or tubing 304 and based on heat transfer principles, estimates the flow rate through the catheter 302 and/or tubing 304. Such heat transfer may be based on temperature of the blood due to body temperature. For example, normal or stable flow through the catheter 302 and/or tubing 304 may measure approximately 37° C. and a reduced temperature when there is no or limited flow through the catheter 302 and/or tubing 304 due to a clog.

The computer system 310 may be communicably coupled to the flow sensor 360 such that it can receive an output signal or data from the flow sensor 360. Further, the computer system 302 can modulate the vacuum pressure and/or aspiration flow rate at the catheter tip (e.g., via a control element 314) in response thereto. For example, if the computer system 310 senses that the flow rate has dropped below a threshold, the computer system 310 can determine that a clog has occurred (e.g., in the tubing 304 or in the catheter 302) and modulate the vacuum pressure and/or aspiration flow rate at the tip of the catheter 302) accordingly (e.g., increase suction and/or aspiration flow rate to facilitate the removal of the clog). TABLE 1 sets forth various illustrative outputs of the flow sensor 360, the states that those measurements would correspond to, and the corresponding response that the vascular treatment system 300 can initiate in response thereto, as described in greater detail below. These values are simply provided to illustrate potential measurements and responses in some embodiments of the vascular treatment system 300 and should not be understood to be limiting in any way.

TABLE 1

| | | | Response |
|---|---|---|---|
| | | Response | (control element |
| | Output of Flow | (vacuum pressure at the catheter | (e.g., pinch valve) |
| State | Sensor | tip) | position) |
| Normal or stable | 20-50 mL/min | Maintain vacuum level | 10% open |
| flow | | | |
| Clog | Decrease to below | Approximately 20-100 torr to be | 25% open |
| | 20 mL/min | applied | |
| Clog release | Increase to over 30 | Approximately 120-750 torr to be | 10% open |
| | mL/min | applied | |

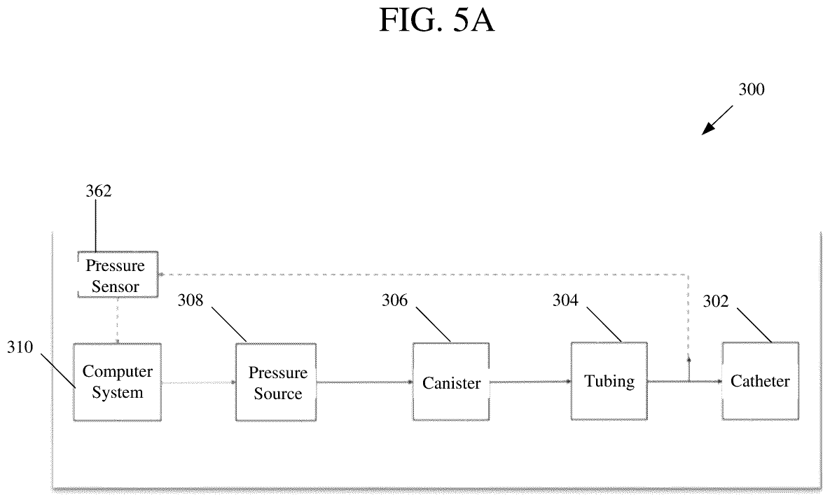

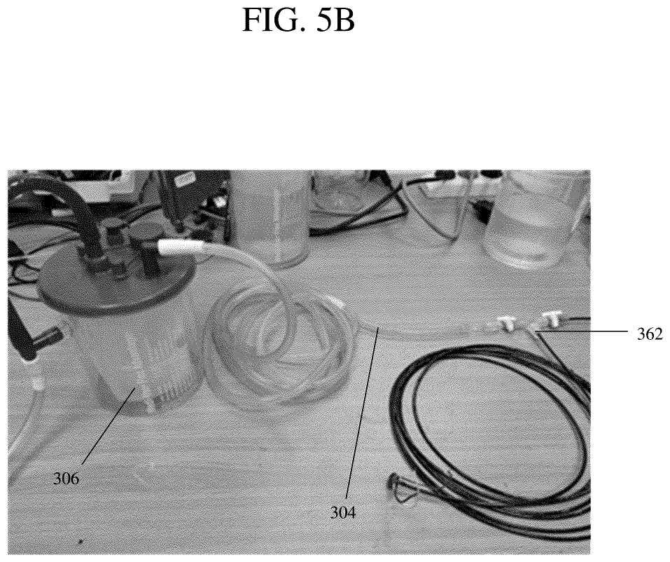

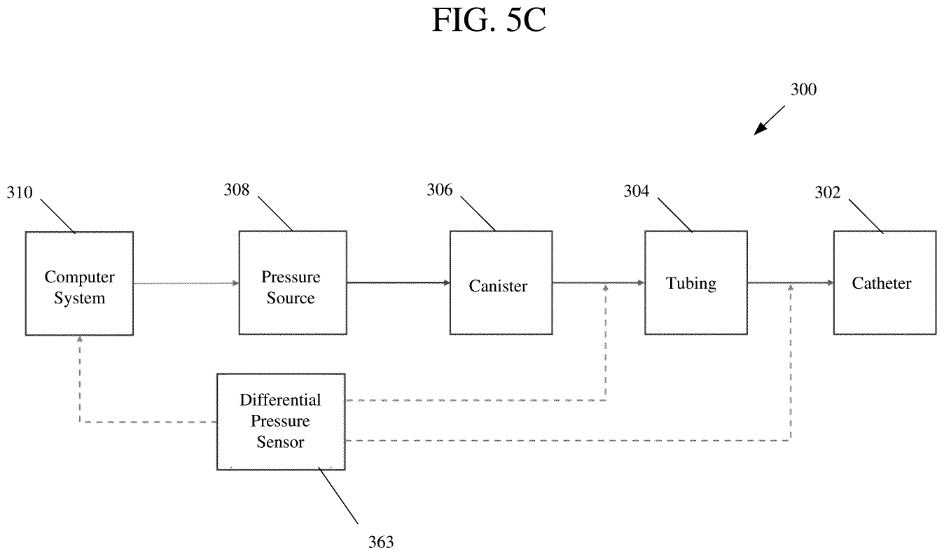

FIGS. 5A and 5B illustrate an embodiment of a vascular treatment system 300 where the sensor 312 includes a pressure sensor 362. The pressure sensor 362 can be configured to sense the vacuum pressure within the tubing 304, catheter 302, or other components of the vascular treatment system 300. The pressure sensor 362 could be positioned at different locations along or within the vascular treatment system 300. In one embodiment, the pressure sensor 362 could be coupled to or along the tubing 304 connecting the canister 306 to the catheter 302 to sense the vacuum pressure within the tubing 304. In another embodiment, the pressure sensor 362 could be configured to sense the vacuum pressure within the catheter 302. For example, the pressure sensor 362 could be placed directly within or adjacent to the handle of the catheter 302. As another example, the pressure sensor 362 could be positioned externally to the catheter 302 and coupled to the handle of the catheter 302 via tubing. In an illustrative embodiment shown in FIG. 5B, the pressure sensor 362 is coupled to the tubing 304 at a distal end thereof adjacent to the connection point to the catheter 302. In other embodiments, the pressure sensor 362 can be positioned in a line parallel to the catheter 302 and/or tubing 304.

The computer system 310 can be communicably coupled to the pressure sensor 362 such that it can receive an output signal or data from the pressure sensor 362. Further, the computer system 310 can modulate the vacuum pressure and/or aspiration flow rate at the catheter tip (e.g., via a control element 314) in response thereto. If the aspiration flow is stable, the pressure within the catheter 302 and/or tubing 304 may remain at a relatively steady state. However, if a clog occurs, the vacuum pressure level may suddenly drop. Therefore, the vacuum pressure dropping below a threshold value or the rate of change of the vacuum pressure dropping by at least a threshold value can be indicative of a clog in the catheter 302 and/or the tubing 304 and a drop in the aspiration flow rate. For example, if the computer system 310 senses that the vacuum pressure has dropped below a threshold value, the computer system 302 can determine that a clog has occurred (e.g., in the tubing 304 or in the catheter 302) and modulate the vacuum pressure and/or aspiration flow rate at the tip of the catheter 302) accordingly (e.g., increase suction and/or aspiration flow rate to facilitate the removal of the clog). TABLE 2 sets forth various illustrative outputs of the pressure sensor 362, the states that those measurements would correspond to, and the corresponding response that the vascular treatment system 300 can initiate in response thereto, as described in greater detail below. These values are simply provided to illustrate potential measurements and responses in some embodiments of the vascular treatment system 300 and should not be understood to be limiting in any way.

TABLE 2

| | | Response | Response |
|---|---|---|---|
| | Output of Pressure | (vacuum pressure at the | (control element (e.g., |
| State | Sensor | catheter tip) | pinch valve) position) |
| Normal OR | 120-750 torr | Maintain vacuum level | Approximately 10% |
| stable flow | | | open |
| Clog | Decrease in pressure at a | Approximately 20-100 torr to | Approximately 25% |
| | rate of approximately equal | be applied | open |
| | to or over 15 torr/second | | |
| Clog release | Increase of pressure at a | Approximately 120-750 torr to | Approximately 10% |
| | rate of approximately equal | be applied | open |
| | to or over 10 torr/second | | |

In another embodiment shown in FIG. 5C, the pressure sensor 362 could include a differential pressure sensor 363. The differential pressure sensor 363 could be coupled at or to two or more components of the vascular treatment system 300 and be configured to measure the vacuum pressure differential between the two or more components of the vascular treatment system 300. In the depicted embodiment, the differential pressure sensor 363 could include a first input coupled between the canister 306 and the tubing 304 and a second input coupled between the tubing 304 and the catheter 302. In other embodiments, the inputs of the differential pressure sensor 363 could be coupled at or to other locations or components of the vascular treatment system 300. Accordingly, the differential pressure sensor 363 could sense the pressure differential between these two locations within the vascular treatment system 300. As with the embodiment described above in connection with FIGS. 5A and 5B, the differential pressure sensor 363 could be coupled to the computer system 310 such that the computer system 310 can receive a signal and/or measurement data therefrom. Accordingly, if the computer system 310 determines that the pressure differential is at or about zero (i.e., there is no pressure differential between two or more of the sensors of the sensor assembly 364), the computer system 310 can determine that a clog has occurred and modulate the vacuum pressure and/or aspiration flow rate at the catheter tip accordingly.

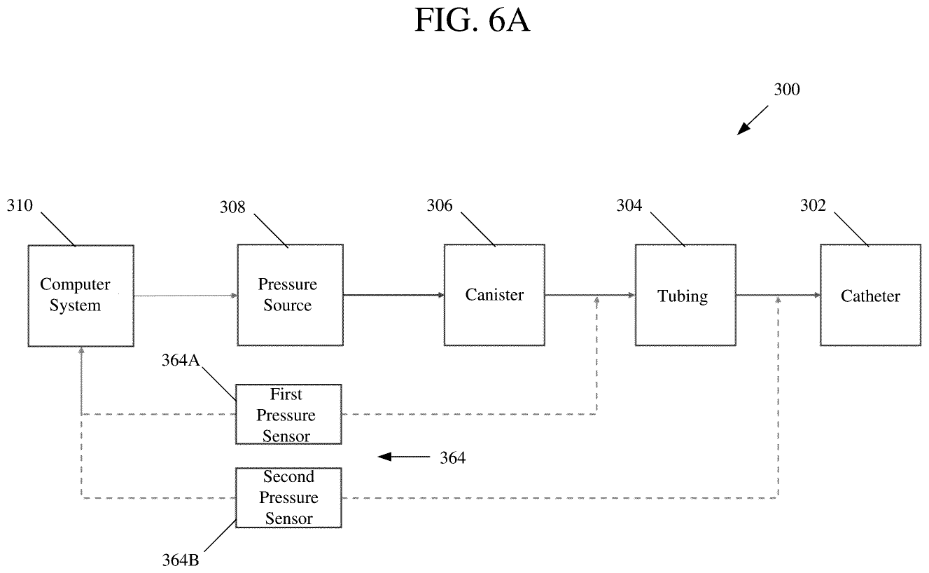

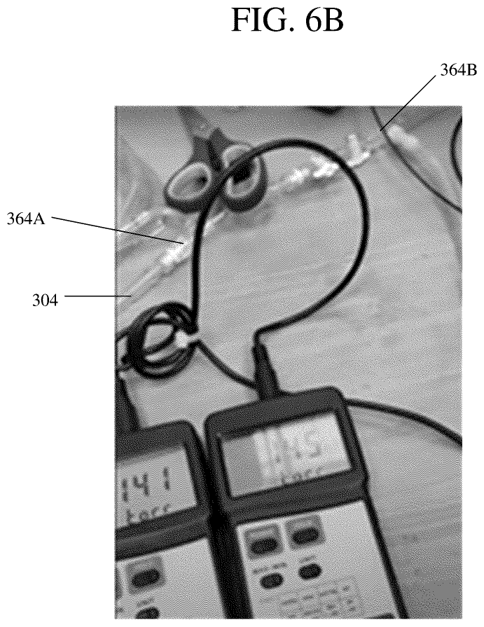

FIGS. 6A and 6B illustrate an embodiment of a vascular treatment system 300 where the sensor 312 includes a pressure sensor assembly 364, i.e., a plurality of pressure sensors. In the depicted embodiment, the pressure sensor assembly 364 includes a first pressure sensor 364A configured to sense the vacuum pressure between the canister 306 and the tubing 304 and a second pressure sensor 364B configured to sense the vacuum pressure between the tubing 304 and the catheter 302. In other embodiments, the pressure sensor assembly 364 could include different numbers of pressure sensors (i.e., more than two) and/or pressure sensors arranged in other configurations or coupled to other components of the vascular treatment system 300. In this embodiment, the various individual pressure sensors of the sensor assembly 364 could be positioned at the same or different locations throughout the vascular treatment system 300. In an illustrative embodiment shown in FIG. 6B, the pressure sensor assembly 364 can include a first pressure sensor 364A coupled to the tubing 304 at a first or upstream position and a second pressure sensor 364B coupled to the tubing 304 at a second or downstream position. The computer system 310 be communicably coupled to each of the pressure sensors making up the pressure sensor assembly 364 such that it can receive an output signal or data therefrom. In this embodiment, the computer system 310 could individually monitor the sensed pressure at each of the locations or monitor the pressure differential between the various sensors of the sensor assembly 364. For example, if the computer system 310 senses that the vacuum pressure has dropped below a threshold at one or more of the sensors of the sensor assembly 364, the computer system 302 can determine that a clog has occurred (e.g., in the tubing 304 or in the catheter 302) and modulate the vacuum pressure and/or aspiration flow rate at the tip of the catheter 302 accordingly (e.g., increase suction to facilitate the removal of the clog). Alternatively, if the computer system 310 determines that the pressure differential between two or more of the sensors of the sensor assembly 364 is at or about zero (i.e., there is no pressure differential between two or more of the sensors of the sensor assembly 364), the computer system 310 can determine that a clog has occurred and modulate the vacuum pressure at the catheter tip accordingly.

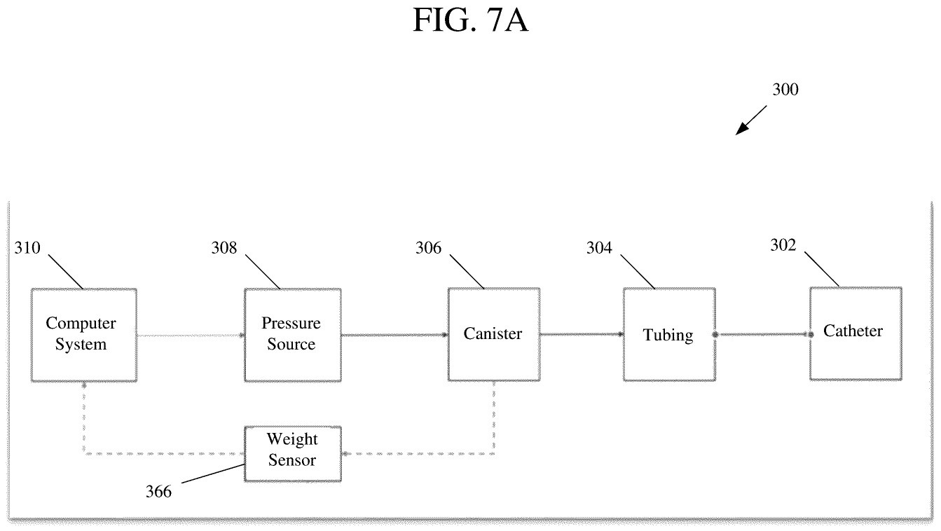

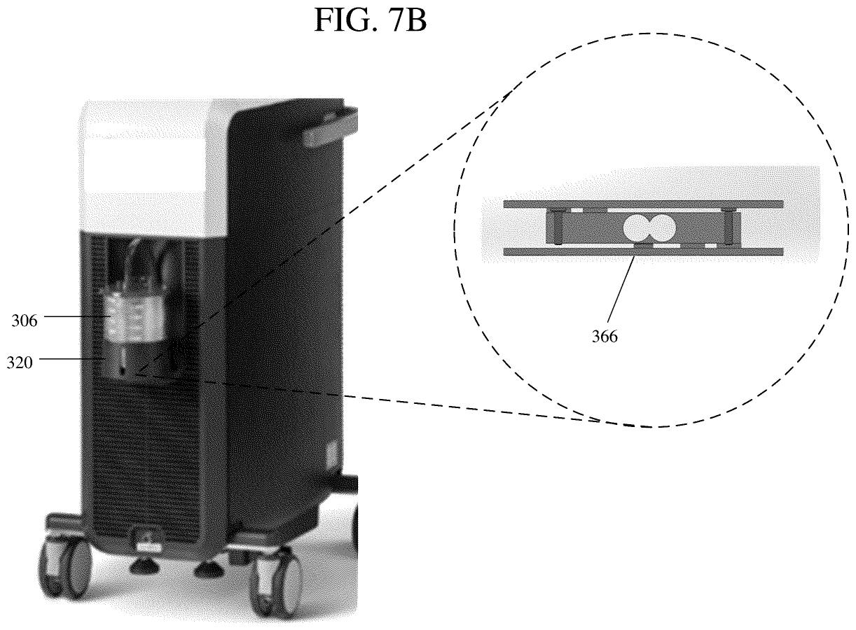

FIGS. 7A and 7B illustrate an embodiment of a vascular treatment system 300 where the sensor 312 includes a weight sensor 366. The weight sensor 366 could include, for example, a load cell. The weight sensor 366 can be configured to sense the weight of the contents within the canister 306. In an illustrative embodiment shown in FIG. 7B, the weight sensor 366 can include a load cell positioned at the base of a holder 320 that is configured to receive the canister 306 such that the base surface of the canister 306 bears against the load cell when placed within the holder 320. Accordingly, the weight sensor 366 can detect the weight of the canister 306 throughout treatment.

The computer system 310 can be communicably coupled to the weight sensor 366 such that it can receive an output signal or data from the weight sensor 366. Further, the computer system 310 can modulate the vacuum pressure and/or aspiration flow rate at the catheter tip (e.g., via a control element 314) in response thereto. Because the change in weight of the canister 306 increases with respect to the flow rate (because the rate at which the weight of the canister 206 is increasing will correspond to the rate at which fluid and/or UIM is being removed from the subject), the computer system 310 can monitor the aspiration flow rate during treatment via the weight of the canister 306. Further, if the rate at which the weight of the canister 306 stops or slows by a threshold amount, the computer system 310 can determine that a clog has occurred and modulate the vacuum pressure and/or aspiration flow rate accordingly. The weight sensor 366 can provide highly accurate estimates for the aspiration flow rate and the total volume of blood that has been aspirated, which can be advantageous because although some other sensor types can identify the occurrence of obstructions with minimal time delay, they may not be able to directly measure the actual aspiration flow rate. Therefore, the weight sensor 366 can be advantageous to incorporate into various embodiments of the vascular treatment system 300. TABLE 3 sets forth various illustrative outputs of the weight sensor 366, the states that those measurements would correspond to, and the corresponding response that the vascular treatment system 300 can initiate in response thereto, as described in greater detail below. These values are simply provided to illustrate potential measurements and responses in some embodiments of the vascular treatment system 300 and should not be understood to be limiting in any way.

TABLE 3

| | | Response | Response |
|---|---|---|---|
| | Output of Weight | (vacuum pressure at the | (control element (e.g., pinch |
| State | Sensor | catheter tip) | valve) position) |
| Normal or | Increase of 20-50 | Maintain vacuum level | Approximately 10% open |
| stable flow | grams/min | | |
| Clog | Increase of under | Approximately 20-100 torr | Approximately 25% open |
| | approximately 15 | to be applied | |
| | grams/min | | |
| Clog release | Increase of | Approximately 120-750 | Approximately 10% open |
| | approximately 30 | torr to be applied | |
| | grams/min | | |

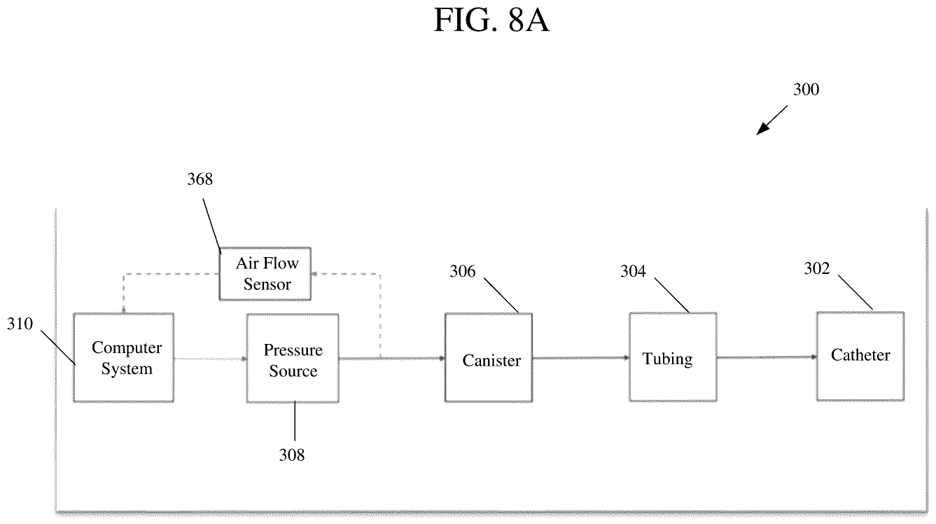

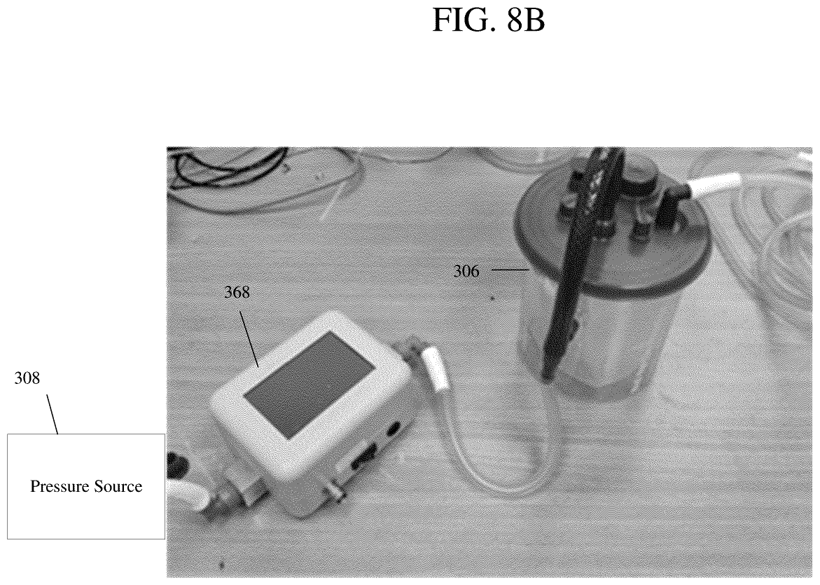

FIGS. 8A and 8B illustrate an embodiment of a vascular treatment system 300 where the sensor 312 includes an air flow sensor 368. The air flow sensor 368 can be configured to sense the air flow rate to the pressure source 308. The air flow sensor 368 can be configured to sense the air flow rate from or within the canister 306 or other components of the vascular treatment system 300. More particularly, the air flow sensor 368 is configured to measure the air flow between the pressure source 308 and the cannister 306. The air flow sensor 368 could be positioned at different locations along or within the vascular treatment system 300. In an illustrative embodiment shown in FIG. 8B, the air flow sensor 368 can be positioned between the pressure source 308 and the canister 306.

The computer system 310 can be communicably coupled to the air flow sensor 368 such that it can receive an output signal or data from the air flow sensor 368. Further, the computer system 310 can modulate the vacuum pressure and/or aspiration flow rate at the catheter tip (e.g., via a control element 314) in response thereto. If the air flow rate stops or decreases by at least a threshold value, that can indicate that there is a clog in the catheter 302 and/or the tubing 304. Accordingly, the computer system 310 can monitor the air flow rate via the air flow sensor 368 and modulate the vacuum pressure and/or aspiration flow rate at the tip of the catheter 302 accordingly. TABLE 4 sets forth various illustrative outputs of the air flow sensor 368, the states that those measurements would correspond to, and the corresponding response that the vascular treatment system 300 can initiate in response thereto, as described in greater detail below. These values are simply provided to illustrate potential measurements and responses in some embodiments of the vascular treatment system 300 and should not be understood to be limiting in any way.

TABLE 4

| | | Response | Response |
|---|---|---|---|
| | | (vacuum pressure at the | (control element (e.g., |
| State | Output of Air Flow Sensor | catheter tip) | pinch valve) position) |
| Normal or stable | 500-4000 mL/min | Maintain vacuum level | Approximately 10% open |
| flow | | | |
| Clog | Decrease in air flow rate of | Approximately 20-100 torr to | Approximately 25% open |
| | approximately 10 mL/min | be applied | |
| Clog release | Increase in air flow of | Approximately 120-750 torr to | Approximately 10% open |
| | approximately 5 mL/min | be applied | |

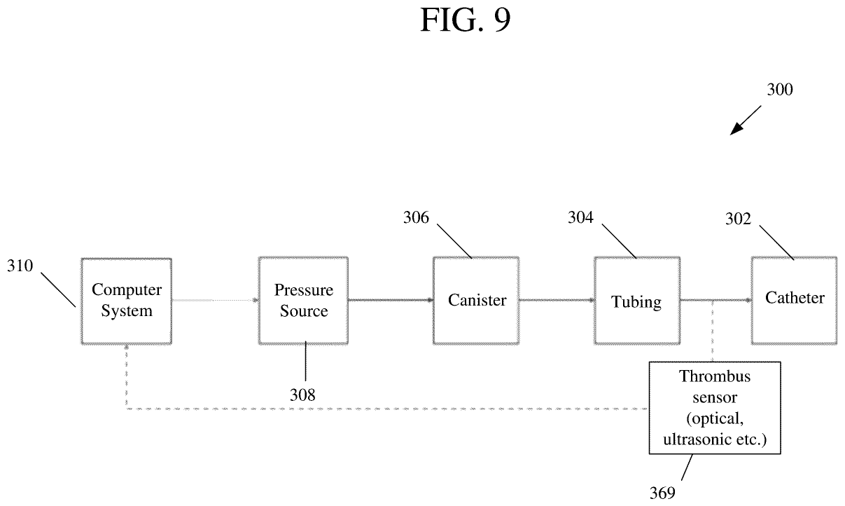

FIG. 9 illustrates an embodiment of a vascular treatment system 300 where the sensor includes a UIM sensor 369. The UIM sensor 369 could include an optical sensor, an ultrasonic sensor, an inductive sensor, a magnetic sensor, a sensor configured to detect electric conductivity, or a turbine sensor, or a variety of other sensors configured to detect the presence of a thrombus or other UIM within the vascular treatment system 300. In one embodiment, the UIM sensor 369 could include an optical sensor, image sensor, or camera that is positioned to identify material that passes through the catheter 302 and/or visually monitor for the presence of UIM (or other obstructions). For example, an optical sensor could be positioned at various locations with respect to the tubing 304 and/or catheter 302. The computer system 310 could then use image analysis techniques on data received from the optical sensor to identify blood and/or UIM passing through the catheter 302. Accordingly, the computer system 310 could modulate the vacuum pressure and/or aspiration flow in response to the identified substances. For example, the computer system 310 could utilize a control element 314 to increase the aspiration flow rate in response to soft thrombus or other UIM being detected via the optical sensor.

Embodiments of the vascular treatment system 300 that are configured to identify thrombi or other UIM could be beneficial because some UIM (particularly, soft thrombi) may flow relatively easily through the vascular treatment system 300 and may not be detected as a clog under certain conditions. However, even if soft thrombi passing through the vascular treatment system 300 are not creating a clog, it would nonetheless be desirable to maintain a high vacuum level and/or aspiration flow rate (e.g., the same or similar vacuum level applied in response to a clog being a detected) to ensure that the soft thrombi are cleared from the tubing 304 and/or catheter 302. Notably, some embodiments described herein monitor the flow rate and total volume aspirated using different sensors 312. However, if a soft thrombus is present, the sensors 312 could mistakenly interpret the thrombus as blood. If a soft thrombus is mistakenly interpreted to be blood by the sensors 312, the computer system 310 could calculate the flow rate and total volume of blood loss incorrectly (i.e., the actual blood loss may be lower than calculated). Therefore, it can be beneficial for some embodiments of the vascular treatment system 300 to further include a UIM sensor 369 to monitor for the presence of soft thrombi or other UIM in order to ensure that the system 300 is properly determining other parameters.

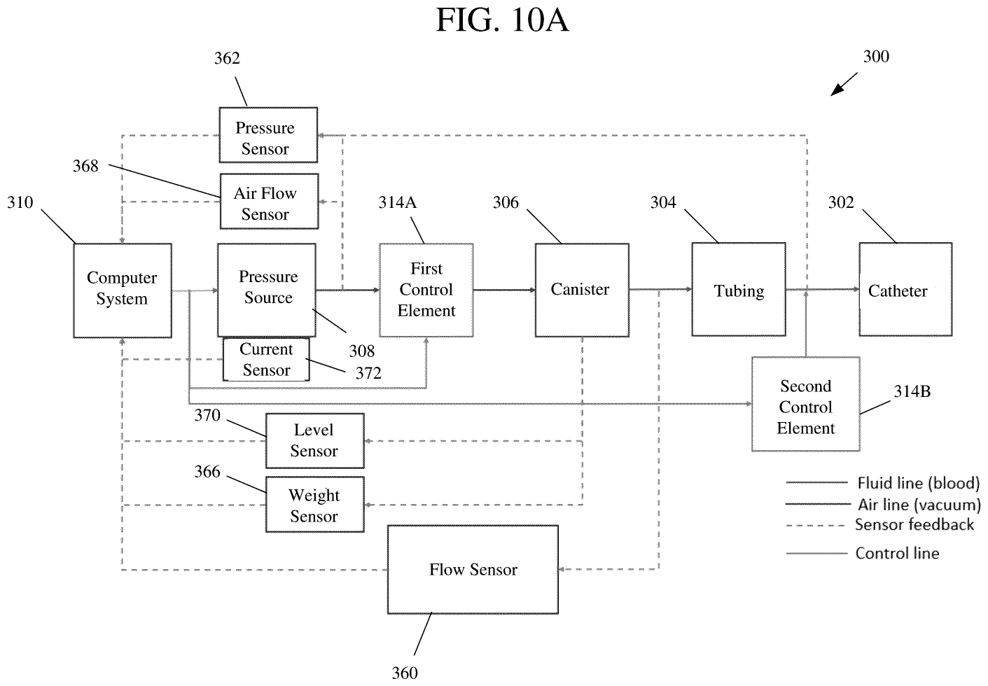

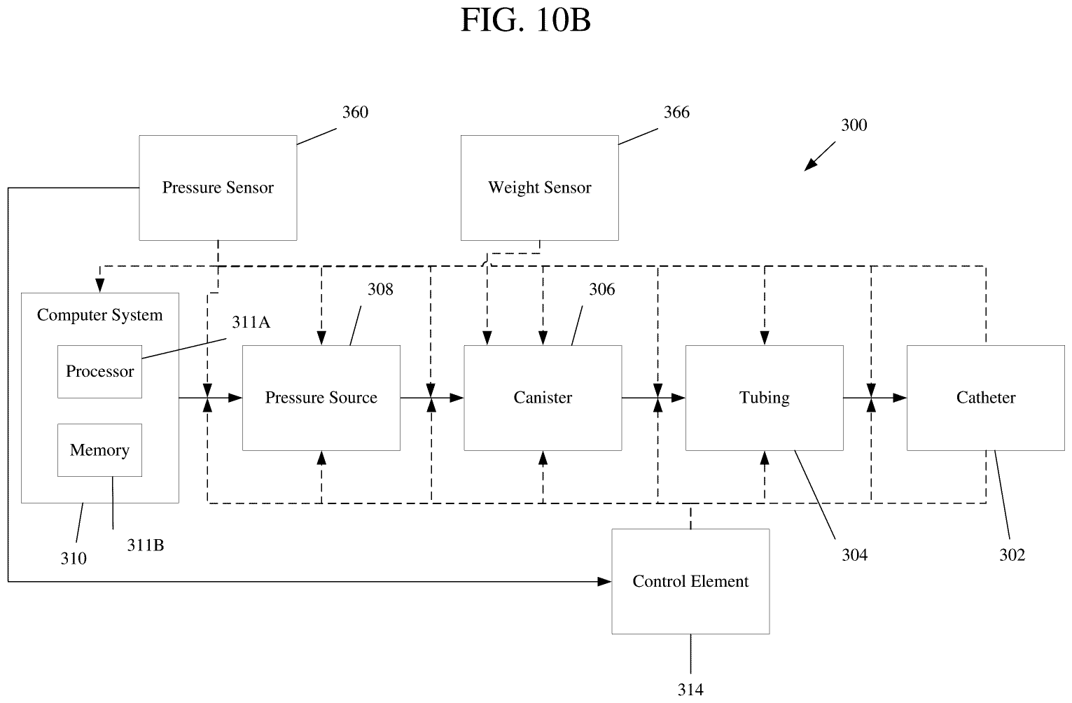

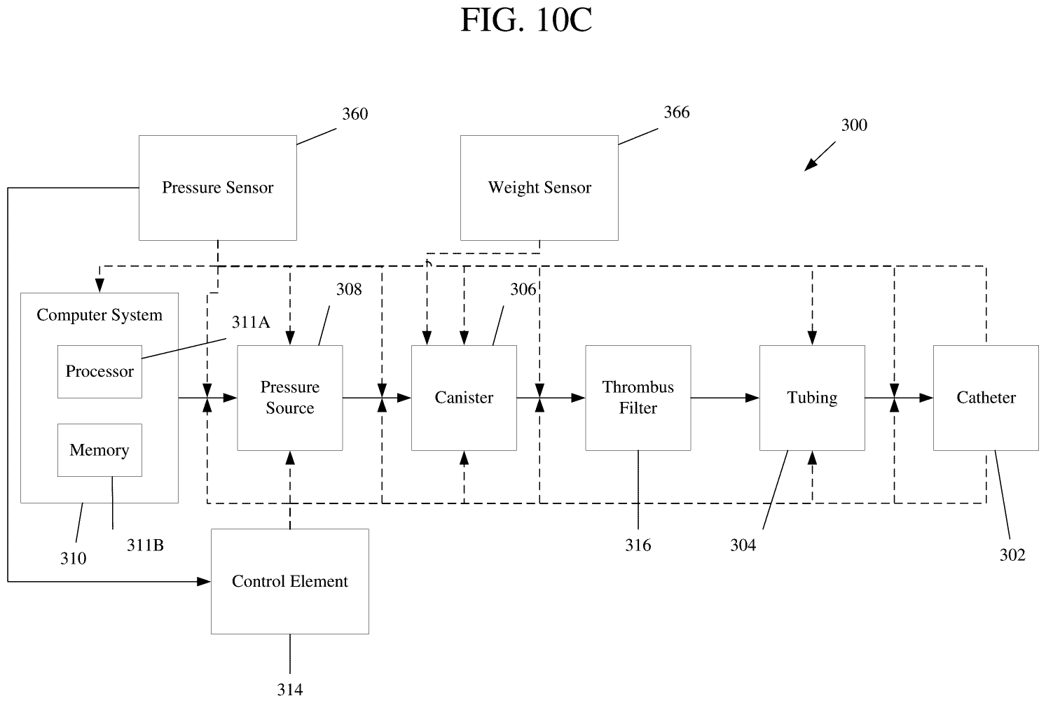

In addition to the sensor types described above, alternative embodiments of the vascular treatment system 300 can include additional sensors, including a level sensor 370 coupled to the canister 306 or a current sensor 372 coupled to the pressure source 308, as shown in FIG. 10A. The level sensor 370 could be used to sense the amount of fluid and/or UIM that has been removed from the subject and is contained within the canister 306. Because the rate at which the amount of fluid and/or UIM in the canister 306 is increasing corresponds to the aspiration flow rate, the computer system 310 could accordingly monitor the aspiration flow rate using the level sensor 370. Further, the current sensor 372 could be configured to monitor the current drawn by the pressure source 308. In some embodiments, the current drawn by the pressure source 308 could correspond to force or torque exerted by a pump motor that is configured to generate the vacuum pressure. Therefore, an increase in the sensed pressure source current could indicate that a clog has occurred because the motor (e.g., a DC motor) could be attempting to compensate for a disruption in the fluid inflow. Therefore, the computer system 310 could accordingly monitor the aspiration flow rate using the current sensor 372. Further, the vascular treatment system 300 could include an aspiration pump that is configured to provide constant flow regardless of the load applied to the system 300 (e.g., the presence of a clog).

Embodiments of the vascular treatment system 300 can include one or more of any of the aforementioned sensors 312 in any combination. For example, FIG. 10A illustrates an embodiment of the vascular treatment system 300 that incorporates all of the sensors described above to sense parameters associated with the system 300 and modulate the aspiration flow accordingly. Further, embodiments of the vascular treatment system 300 can include different sensors 302, either in lieu of the aforementioned sensors or in combination thereof. In particular, it could be advantageous to use various combinations of sensors because each sensor type has its own strength. Further, various embodiments of the vascular treatment system 300 could include multiple control elements 314. For example, the embodiment illustrated in FIG. 10A includes a first control element 314A (e.g., a control valve) interposed between the pressure source 308 for controlling the aspiration flow therethrough and the canister 306 and a second control element 314B (e.g., a proportional pinch valve) configured to control aspiration flow between the tubing 304 and the catheter 302. Various embodiments of the control elements 314 are described in greater detail below.

As another example, FIG. 10B illustrates an embodiment of the vascular treatment system 300 that includes a pressure sensor 362 in combination with a weight sensor 366. In alternative embodiments, the vascular treatment system 300 could include a differential pressure sensor 363 or a pressure sensor assembly 364 in lieu of or in addition to the pressure sensor 362. As described above and indicated in FIG. 10B, the pressure sensor 362 could be coupled to a variety of different components of the vascular treatment system 300 and/or be other configured to sense the internal vacuum pressure between a variety of different components of the vascular treatment system 300. In this embodiment, the pressure sensor 362 can identify blockages with a relatively minimal time response. Further, the weight sensor 366 for the canister 306 can provide highly accurate estimates for the aspiration flow rate, but has a relatively high time delay because the canister 306 is downstream from the catheter 302. Therefore, it could be beneficial for embodiments of the vascular treatment system 300 to use a combination of the pressure sensor 362 and the weight sensor 366 in order to both quickly identify blockages and obtain highly accurate measurements of the aspiration flow rate. Additional embodiments of the vascular treatment system 300 could utilize other combinations of sensor types to obtain different benefits or simply provide measurement redundancy from the different sensor types.

As discussed above, the one or more sensors 312 can be operably coupled to a control element 314 that is configured to modulate the vacuum pressure generated through the catheter 302. Embodiments of the vascular treatment system 300 can include a variety of different control elements 314 that can be configured to control the computer system 310, the pressure source 308, the canister 306, the tubing 304, the catheter 302, or any of the connections between the aforementioned components to modulate the vacuum pressure at the catheter tip for the vascular treatment system 300.

Referring now to FIG. 10C, there is shown an embodiment of the vascular treatment system 300 include a UIM filter 316. As noted above with respect to the embodiment illustrated in FIG. 9, it can be beneficial for the vascular treatment system 300 to identify the presence of UIM, such as soft thrombi or UIM fragments. However, it could be further beneficial for the vascular treatment system 300 to remove soft thrombi from the fluid lines of the system 300. The UIM filter 316 could include a filter or barrier that is configured to capture a soft thrombus or other UIM. In the embodiment illustrated in FIG. 10C, the vascular treatment system 300 can include a UIM filter 316 positioned in-line between the catheter 302 and the canister 306. As an example, the UIM filter 316 could be positioned before a pressure sensor 360 so that any soft thrombi are prevented from reaching the pressure sensor 360 and, thus, would not affect any measurements by the pressure sensor 360. In another embodiment, the UIM filter 316 could be positioned within the canister 306. In this embodiment, the UIM filter 316 could be coupled to a separate weight sensor (i.e., a distinct weight sensor from the weight sensor 366) that is configured to weigh a thrombus captured by the UIM filter 316. The computer system 310 could further be coupled to the UIM filter weight sensor (not shown). Accordingly, the computer system 310 could subtract the weight of the captured thrombus (e.g., as determined by the UIM filter weight sensor) from the total weight of the canister 306 (e.g., as determined by the weight sensor 366). In yet another embodiment, the vascular treatment system 300 could include a sensor configured to detect a level of material within the canister 306 (e.g., an ultrasonic level sensor), which can in turn be utilized by the computer system 310 to determine the volume and flow rate of the aspirated fluid remove from the subject, without the UIM (e.g., a soft thrombus) being present within the canister 306 to affect those calculations. In these embodiments, the computer system 310 could compensate for the presence of a UIM and thereby accurately determine various parameters associated with the system 300, such as the actual blood loss by the subject. In some embodiments, these data or parameters could further be provided to the user by the computer system 310. In some embodiments, despite the weight sensor 366 being present, the computer system 310 can ignore changes detected by the weight sensor 366, such as, for example, when there is a reduction in weight (which would not be reasonable) or if there is a large sudden weight change because of mechanical force applied on the cannister 306.

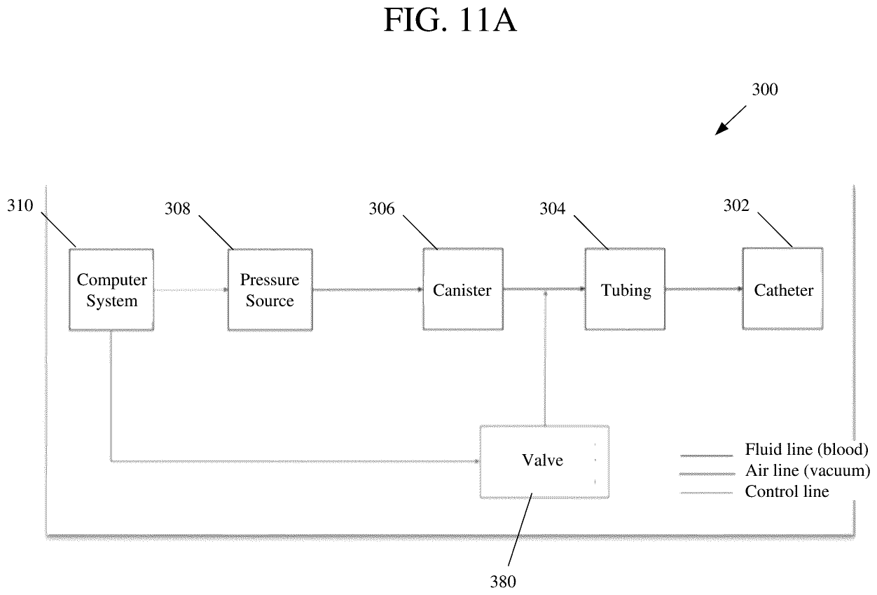

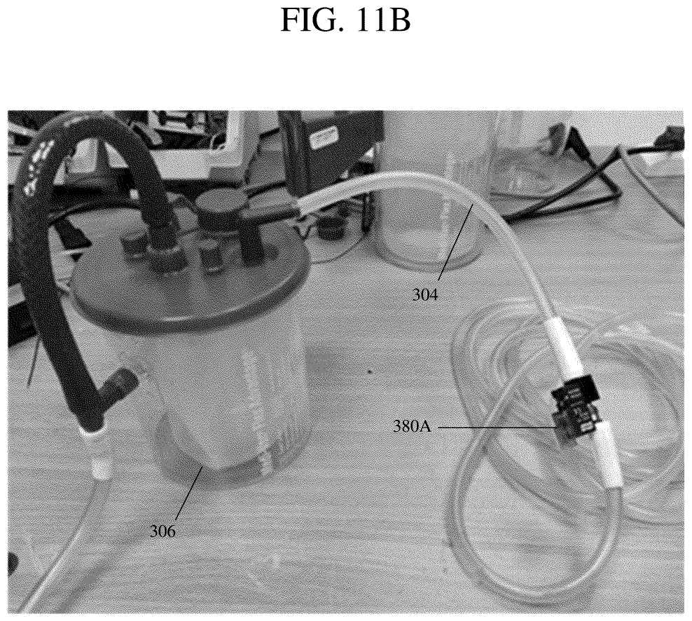

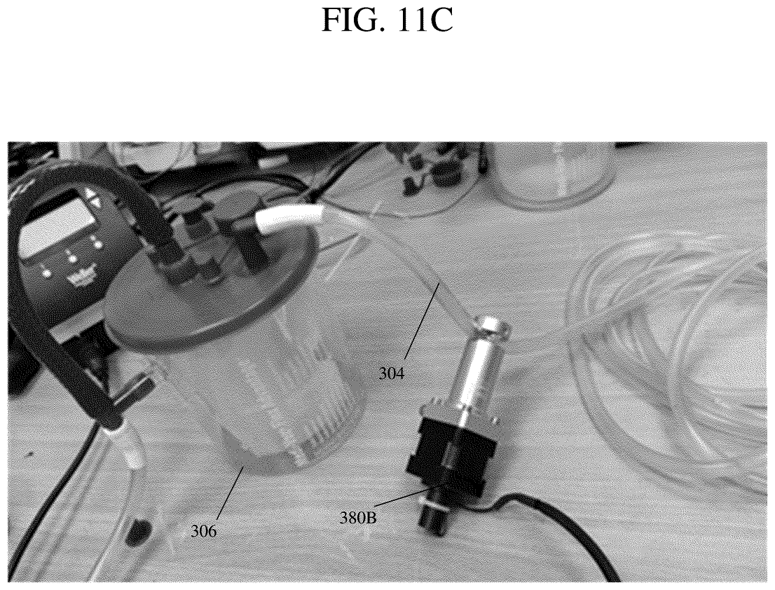

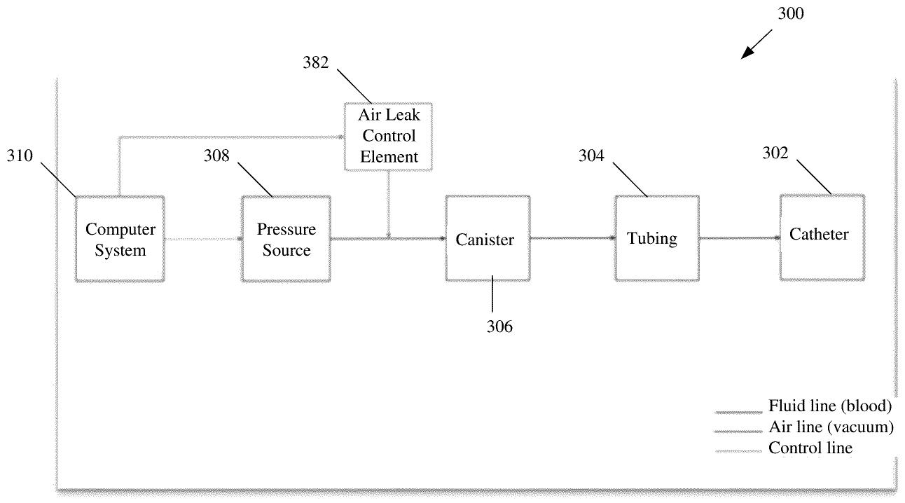

FIGS. 11A-C illustrate an embodiment of a vascular treatment system 300 where the control element 314 includes a valve 380 configured to modulate the vacuum pressure and/or aspiration flow rate at the catheter tip therethrough. The valve 380 can be positioned at various locations within the vascular treatment system 300 for modulating the aspiration flow therethrough, including being coupled to the tubing 304 connecting the catheter 302 to the canister 306. The valve 380 can include a proportional valve, a solenoid valve, a pinch valve, or a gate. In an illustrative embodiment shown in FIG. 11B, the valve 380 includes a modulated solenoid valve 380A that is placed on the catheter tubing 304, adjacent to the canister 306. In another illustrative embodiment shown in FIG. 11C, the valve 380 includes a pinch valve 380B that is positioned similarly to the embodiment shown in FIG. 11B. In one embodiment, the valve 380 can be controlled by the computer system 310 to open and close according to a duty cycle, which is set by the computer system 310 in response to the sensed aspiration flow state. For example, the duty cycle of the valve 380 could be modulated to increase the vacuum pressure and/or aspiration flow rate at the catheter tip when the computer system 310 detects that a clog may be present (e.g., via the sensor 312). In yet another illustrative embodiment, the valve 380 could include a proportional valve and the amount or degree to which the valve is closed could be controlled by the computer system 310. Accordingly, the computer system 310 could modulate the vacuum pressure and/or aspiration flow rate at the tip of the catheter 302 by controlling the amount or degree to which the proportional valve is opened or closed in response to the sensed aspiration flow state.

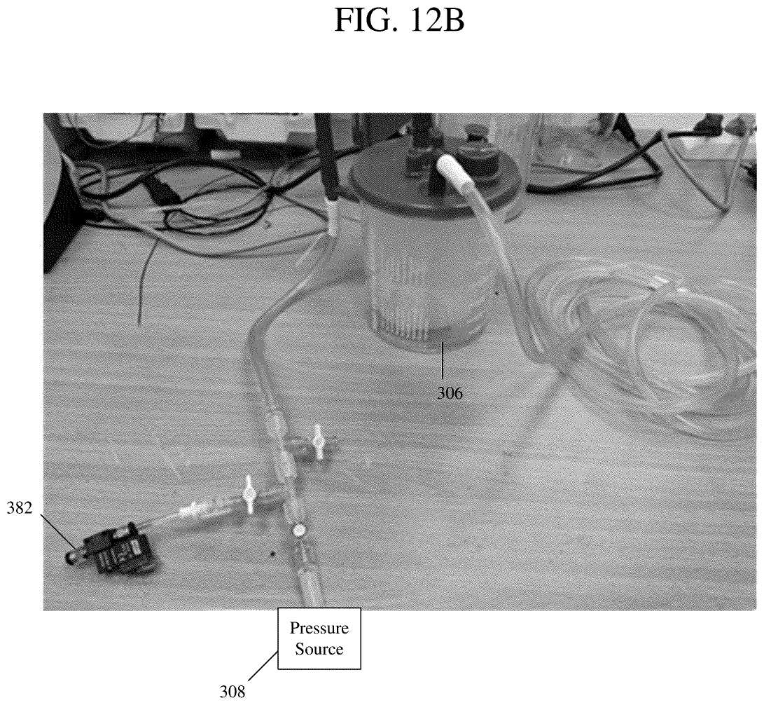

FIGS. 12A and 12B illustrate an embodiment of a vascular treatment system 300 where the control element 314 is configured to utilize an air leak control element 382 to modulate the vacuum pressure at the catheter tip. The air leak control element 382 is configured to controllably permit air to enter the vascular treatment system 300 in order to modulate the generated vacuum pressure at the catheter tip. In various embodiments, the aperture of an air leak control element 382 could range from about 0.3 mm to about 0.6 mm. By allowing air in the system 300, the air leak control element 382 can effectively control the strength of the vacuum pressure experienced by the catheter 302, which in turn affects the aspiration flow rate generated thereby. In other words, as more air is permitted to enter the vascular treatment system 300, the vacuum pressure may decrease. By controlling the degree of air leakage through the air leak control element 382, the computer system 310 could accordingly control the aspiration flow rate. The air leak control element 382 could include a variety of different valves, nozzles or gates configured to permit air inflow therethrough. In an illustrative embodiment shown in FIG. 12B, the air leak control element 382 includes a solenoid valve that is positioned at a junction between the pressure source 308 and the canister 306 to modulate the vacuum pressure generated by the pressure source 308. In one embodiment, the air leak control element 382 can be controlled by the computer system 310 to open and close according to a duty cycle, which is set by the computer system 310 in response to the sensed aspiration flow state. For example, the duty cycle of the air leak control element 382 could be modulated to increase the vacuum pressure at the catheter tip (i.e., decrease the air leakage inflow, which in turn increases the vacuum pressure) when the computer system 310 detects that a clog may be present (e.g., via the sensor 312). In another embodiment, the aperture size of the air leak control element 382 could be controllable in response to the sensed aspiration flow state. For example, the aperture size of the air leak control element 382 could be decreased to increase the vacuum pressure at the catheter tip (i.e., decrease the air leakage inflow, which in turn increases the vacuum pressure) when the computer system 310 detects that a clog may be present (e.g., via the sensor 312).

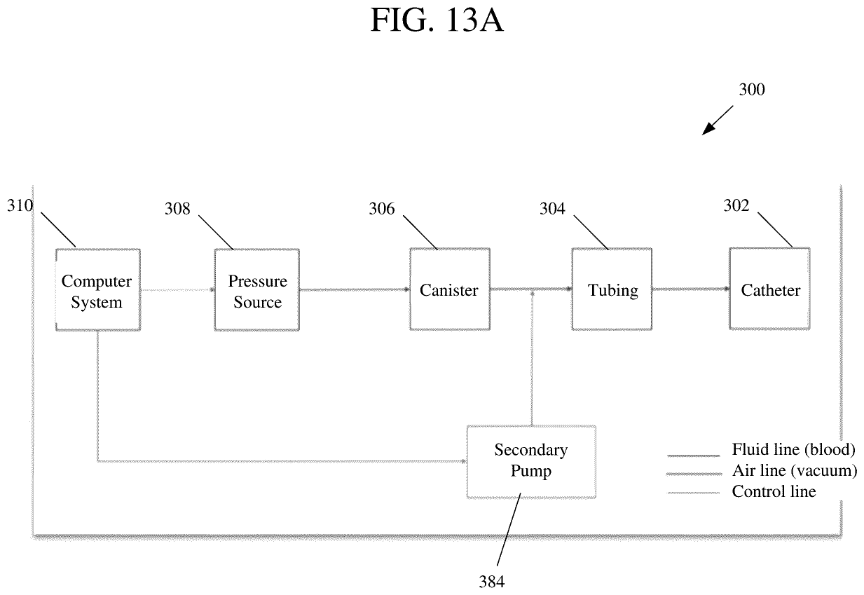

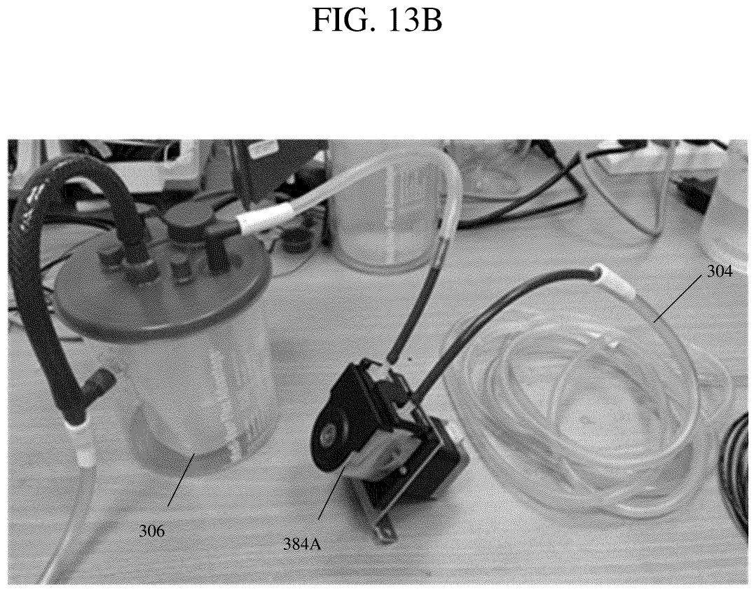

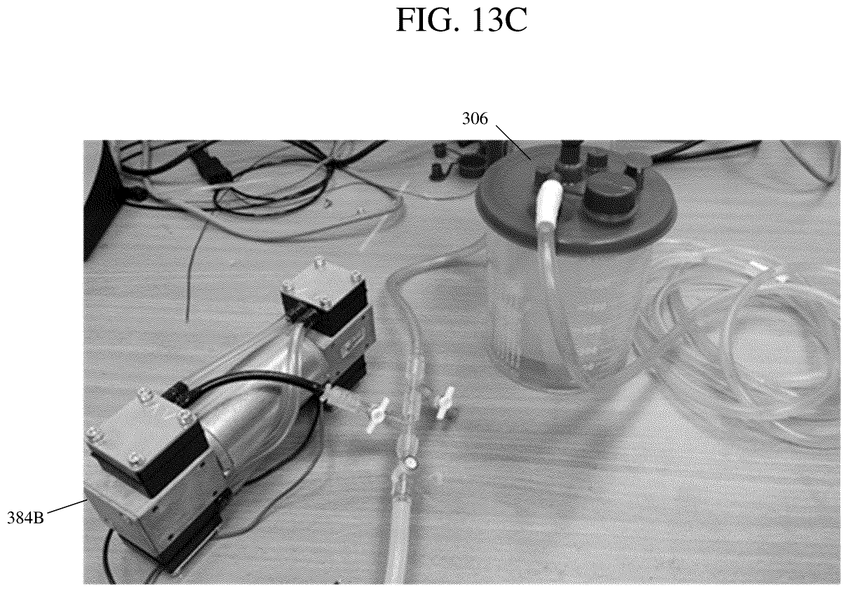

FIGS. 13A-C illustrate embodiments of a vascular treatment system 300 where the control element 314 includes a secondary pump 384 configured to modulate the vacuum pressure at the catheter tip therethrough. The pump 384 could differ from the pressure source 308 described above. The pump 384 can be positioned at various locations within the vascular treatment system 300 for modulating the aspiration flow, including being coupled to the tubing 304 connecting the catheter 302 to the canister 306. In an illustrative embodiment shown in FIG. 13B, the pump 384 includes a peristaltic pump 384A that is coupled to the catheter tubing 304 adjacent to the canister 306. In another illustrative embodiment shown in FIG. 13C, the pump 384 includes a DC pump 384B that is coupled to the catheter tubing 304 adjacent to the canister 306. The pump 384 can be configured to pump fluid and/or UIM at varying speeds as determined by the computer system 310 in response to the sensed aspiration flow state. For example, the speed of the pump 384 could be increased, which in turn increases the vacuum pressure at the catheter tip, when the computer system 310 detects that a clog may be present (e.g., via the sensor 312).

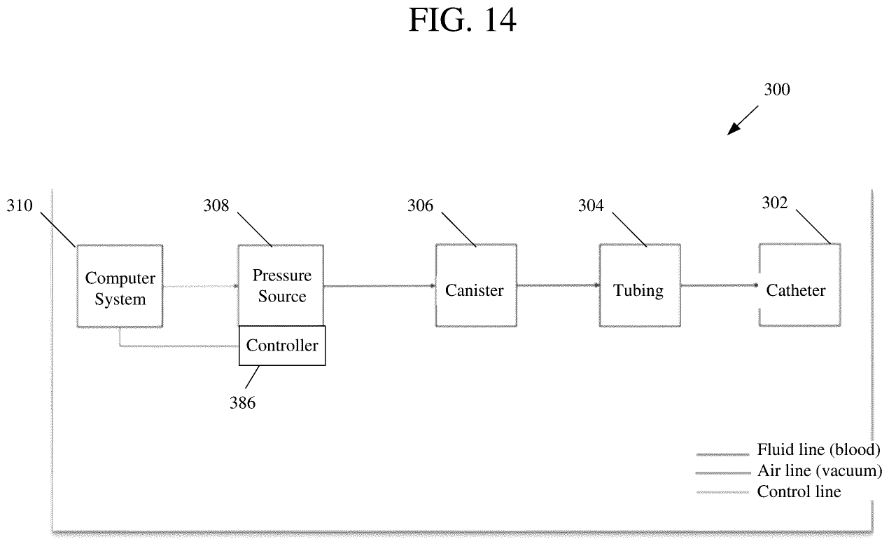

FIG. 14 illustrates an embodiment of a vascular treatment system 300 where the control element 314 includes a controller 386 configured to directly control the pressure source 308 to modulate the aspiration flow. The controller 386 could include a variety of different hardware, software, and/or firmware controllers that are configured to control the output of a pressure source 308 (e.g., a pump). In some embodiments, the controller 386 could include a pulse width modulation (PWM) or voltage controller. In one illustrative embodiment, the pressure source 308 could be controlled by the computer system 310 via a PWM signal or direct voltage control to control the speed of the pressure source 308. The controller 386 could control the pressure source 308 to modulate the vacuum pressure at the catheter tip as generated thereby as determined by the computer system 310 in response to the sensed aspiration flow state. For example, the controller 386 could increase the speed (e.g., rotations per minute) of the pump when the computer system 310 detects that a clog may be present (e.g., via the sensor 312). In another embodiment where the pressure source 308 includes a multi-headed pump, the computer system 310 could modulate the arrangement of the pump heads via the controller 386. In particular, when it is desired to increase the vacuum level (e.g., in response to a clog being detected), the pump heads could be connected in series with each other. Conversely, when it is desired to decrease the vacuum level, the pump heads could be connected in parallel.

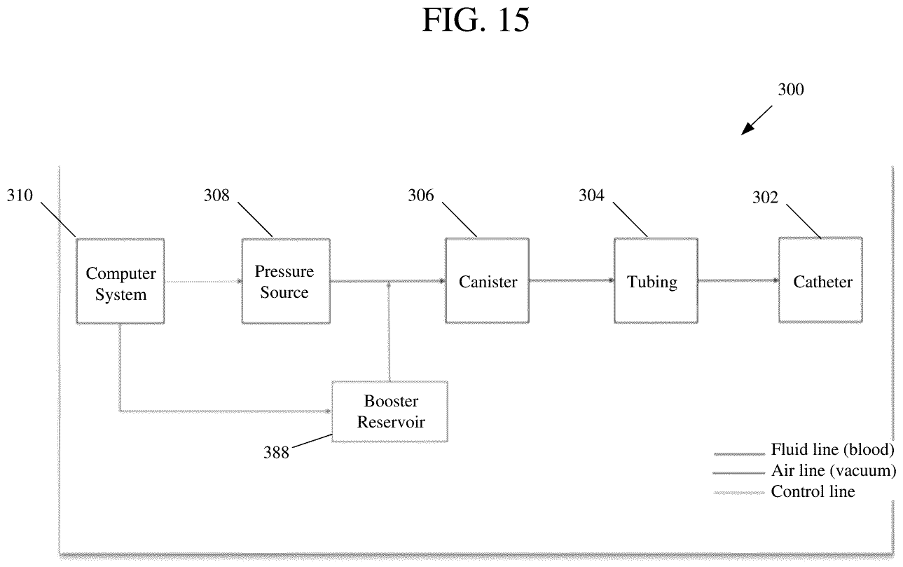

FIG. 15 illustrates an embodiment of a vascular treatment system 300 where the control element 314 includes a booster reservoir 388 that can be utilized to modulate the aspiration flow. In this embodiment, the booster reservoir 388 could be used as an additional source of vacuum pressure that can be selectively coupled to the canister 306 and/or another component of the vascular treatment system 300 in order to modulate the vacuum pressure at the catheter tip. In particular, the booster reservoir 388 could be coupled to the vascular treatment system 300 in order to increase the vacuum pressure of the system 300, which in turn would increase the vacuum pressure at the catheter tip and thereby allow for obstructions to be cleared as necessary. The booster reservoir 388 could be coupled to one or more of the other components of the vascular treatment system 300 (e.g., the catheter tubing 304) via a valve or another device that allows for the access to the booster reservoir 388 to be controlled, which in turn dictates the amount by which the generated vacuum pressure is modulated thereby. In an illustrative embodiment shown in FIG. 15B, the booster reservoir 388 is coupled to the catheter tubing 304, adjacent to the canister 306, via a solenoid valve. The computer system 310 can be configured to control the solenoid valve in response to the sensed aspiration flow state in order to control the amount of additional vacuum pressure that is provided by the booster reservoir 388, which in turn affects the vacuum pressure experienced at the catheter tip. For example, when the computer system 310 detects that a clog may be present (e.g., via the sensor 312), the computer system 310 could control the valve to couple the booster reservoir 388 to the canister 306 to increase the vacuum pressure in order to clear the clog. In one embodiment, the booster reservoir 388 could be coupled to the vascular treatment system 300 via a proportional valve or another connector to allow the amount of supplemental vacuum pressure provided by the booster reservoir 388 to be modulated by controlling the amount or degree to which the valve is opened.

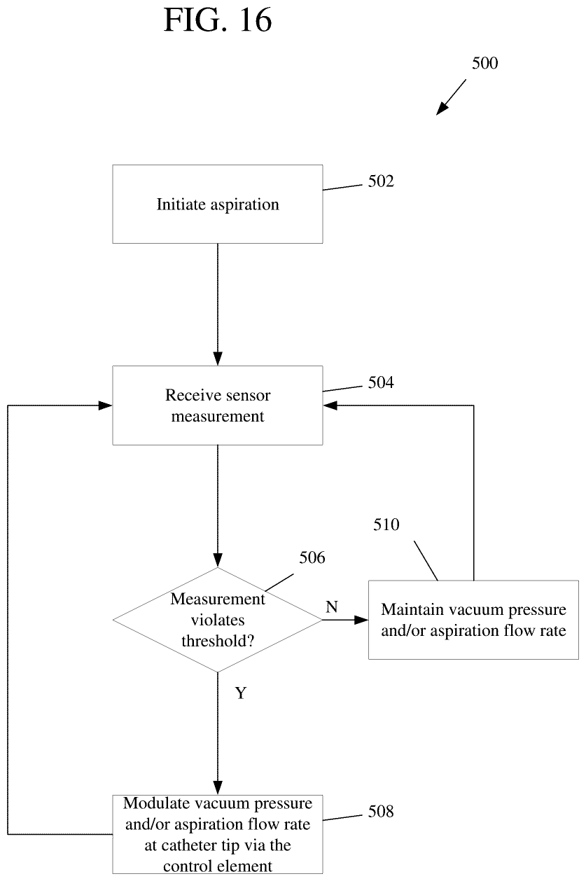

As described throughout, the vascular treatment system 300 can sense various parameters associated with the system 300 and modulate the aspiration flow rate generated through the catheter 302 accordingly. Various embodiments processes 500, 600, 700 for modulating the aspiration flow rate are shown in FIGS. 16-18. In one embodiment, the processes 500, 600, 700 can be embodied as instructions stored in a memory (e.g., the memory 311B) that, when executed by a processor (e.g., the processor 311A), causes the computer system 310 to perform the processes 500, 600, 700. In various embodiments, the processes 500, 600, 700 can be embodied as software, hardware, firmware, and various combinations thereof. In various embodiments, the processes 500, 600, 700 can be executed by and/or between a variety of different devices or systems. For example, various combinations of operations of the processes 500, 600, 700 could be executed by the computer system 310 and/or other components of the vascular treatment system 300. In various embodiments, the computer system 310 executing the process 500 can utilize distributed processing, parallel processing, cloud processing, and/or edge computing techniques. For brevity, the processes 500, 600, 700 are described below as being executed by the computer system 310; however, it should be understood that the functions can be individually or collectively executed by one or multiple devices of the vascular treatment system 300.

Referring now to FIG. 16, there is shown one embodiment of a process 500 for modulating the aspiration flow. Accordingly, the computer system 310 executing the process 500 can cause the vascular treatment system 300 to initiate 502 aspiration through the catheter 302 (e.g., by activating the pressure source 308). During aspiration, the computer system 310 can receive 504 a sensor measurement from one or more of the sensors 312. The received sensor measurement could include, for example, direct (e.g., via a flow sensor 360 or pressure sensor 362) or indirect (e.g., via a canister weight sensor 366) measurements of the aspiration flow rate. However, received sensor measurements could be from any of the sensors discussed herein. Further, the computer system 310 can determine 506 whether the received sensor measurement violates a threshold (e.g., exceeds a threshold value or falls below a threshold value). The threshold could include a default value, a range of values, a baseline measurement for the vascular treatment system 300, a rate of change of the sensed parameter (or another derived measurement), and so on. If the sensor measurement violates the threshold, the computer system 310 can modulate 508 the vacuum pressure and/or aspiration flow rate at the catheter tip using the control element 314 accordingly. For example, if the sensed flow rate falls below a threshold value, the computer system 310 can utilize the control element 314 to increase the vacuum pressure and/or aspiration flow rate at the catheter tip. Conversely, if the sensed measurement does not violate the threshold, the computer system 310 can take no action or otherwise maintain 510 the vacuum pressure (thereby maintaining the present aspiration flow rate). In one embodiment, the level at which the aspiration flow and/or vacuum pressure are maintained 510 could be dependent upon the state of a secondary device associated with or within the vascular treatment system 300. For example, if the laser of the catheter 302 is activated, the vacuum pressure and/or aspiration flow rate could be maintained at a first level; conversely, if the laser is not activated, the vacuum pressure and/or aspiration flow rate could be maintained at a second level. In some embodiments, when the laser is not activated, and before the procedure starts the control element 314 can restrict the aspiration flow rate completely (i.e., no flow). As indicated in FIG. 15, regardless of whether the computer system 310 modulates 508 or maintains 510 the vacuum pressure at the catheter tip, the computer system 310 can continue receiving 504 sensor measurements and acting accordingly to control the vacuum pressure and/or aspiration flow rate at the catheter tip.

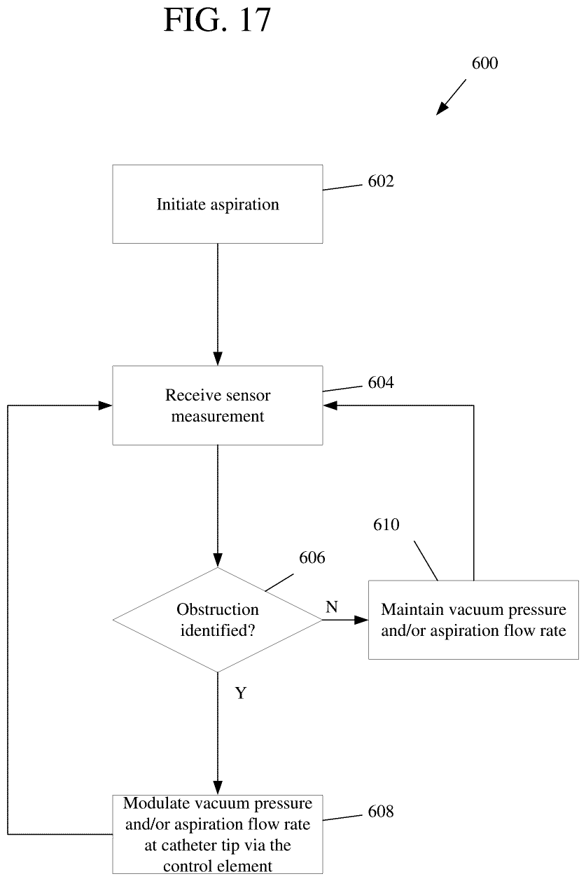

Referring now to FIG. 17, there is shown another embodiment of a process 600 for modulating the aspiration flow. Similarly, as described above with respect to the process 500 depicted in FIG. 16, the computer system 310 executing the process 600 can cause the vascular treatment system 300 to initiate 602 aspiration through the catheter 302 and receive 604 a sensor measurement from one or more of the sensors 312 during the aspiration. In this embodiment, the computer system 310 can determine 606 whether an obstruction is present. The computer system 310 could determine 606 that an obstruction is present either directly (e.g., by UIM being detected by an optical sensor) or indirectly (e.g., based on a drop in the pressure differential across one or more components of the vascular treatment system 300 or the sensed aspiration flow rate dropping below a threshold) via one or more sensors 312, as described throughout the present disclosure. Similarly, to the aforementioned embodiment, if the computer system 310 identifies the presence of an obstruction, the computer system 310 can modulate 608 the vacuum pressure and/or aspiration flow rate at the catheter tip using the control element 314 accordingly (e.g., increasing suction). Conversely, if the computer system 310 does not identify the presence of an obstruction, the computer system 310 can take no action or otherwise maintain 610 the vacuum pressure (thereby maintaining the present aspiration flow rate). As with the aforementioned embodiment, the aspiration flow and/or vacuum pressure could be maintained at different levels depending upon the state of a secondary device associated with or within the vascular treatment system 300. As indicated in FIG. 17, regardless of whether the computer system 310 modulates 608 or maintains 610 the vacuum pressure at the tip of the catheter 302, the computer system 310 can continue receiving 604 sensor measurements and acting accordingly to control the vacuum pressure and/or aspiration flow rate at the catheter tip in response to the detection of any obstructions.

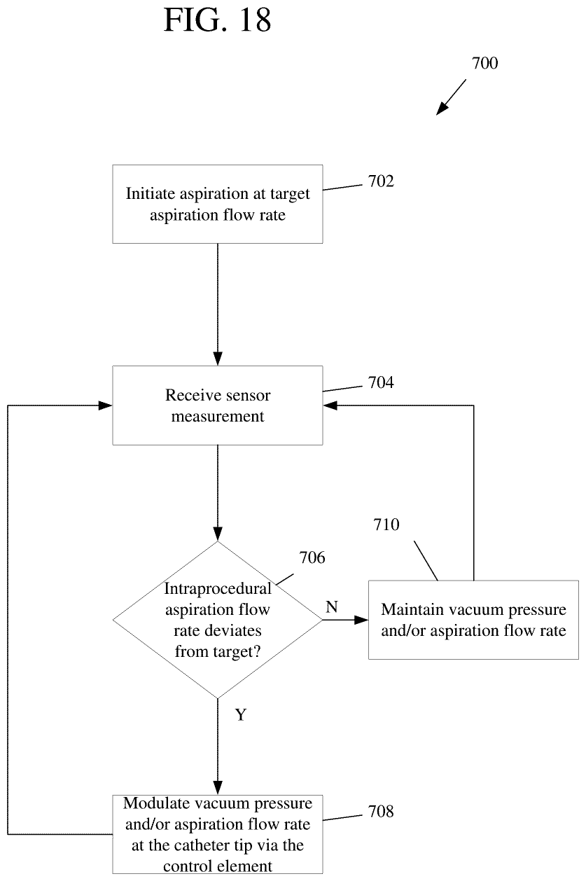

Referring now to FIG. 18, there is shown another embodiment of a process 700 for modulating the aspiration flow. Accordingly, the computer system 310 executing the process 700 can cause the vascular treatment system 300 to initiate 702 aspiration through the catheter 302 at a target aspiration flow rate. In one embodiment, the target aspiration flow rate could be input by a user via a GUI provided by the computer system 310, for example. In one embodiment, the target aspiration flow rate could be automatically determined by the computer system 310 based on the size of the catheter 302. For example, the vascular treatment system 300 could include a sensor (e.g., an RFID reader) that is configured to detect the type of catheter 302 being utilized, which can in turn be used to determine the size of the catheter 302. Based on the size of the catheter 302, the computer system 310 could set the target aspiration flow rate and a variety of other parameters for the system 300 (e.g., vacuum level, vacuum threshold, flow rate threshold, etc.) accordingly. The target aspiration flow rate could be a particular value or a range of values. In one embodiment, the target aspiration flow rate could be about 20 mL/min to about 100 mL/min. In another embodiment, the target aspiration flow rate could be about 20 mL/min to about 50 mL/min. Further, the computer system 310 can receive 704 a sensor measurement from one or more of the sensors 312 during the aspiration and determine 706 whether the intraprocedural aspiration flow rate deviates from the target aspiration flow rate value or range. In one embodiment, the computer system 310 could directly measure the aspiration flow rate (e.g., via a flow sensor 360 or a weight sensor 366) or indirectly measure the aspiration flow rate via one or more sensors 312, as described throughout the present disclosure. If the computer system 310 determines that the intraprocedural aspiration flow rate deviates from the target value or range, the computer system 310 can modulate 708 the vacuum pressure and/or aspiration flow rate at the catheter tip using the control element 314 to bring the intraprocedural flow rate into alignment with the target aspiration flow rate. Conversely, if the computer system 310 determines that the intraprocedural aspiration flow rate does not deviate from the target value or range, the computer system 310 can take no action or otherwise maintain 710 the vacuum pressure at the catheter tip (thereby maintaining the present aspiration flow rate). As with the aforementioned embodiment, the aspiration flow and/or vacuum pressure could be maintained at different levels depending upon the state of a secondary device associated with or within the vascular treatment system 300. As indicated in FIG. 18, regardless of whether the computer system 310 modulates 708 or maintains 710 the vacuum pressure at the catheter tip, the computer system 310 can continue receiving 704 sensor measurements and acting accordingly to control the vacuum pressure at the catheter tip in response to the detected intraprocedural aspiration flow rate.

It can be advantageous for the vascular treatment system 300 to maintain a target aspiration flow rate, regardless of the load being experienced by the system 300 from the aspiration flow, for a number of different reasons. For example, the pressure source 308 can react directly to the load on the system 300 or the pressure source 308 can be optimized for particular restrictions or loads. Accordingly, it can be beneficial for some embodiments of the computer system 310 to execute the process 700.

Referring back to FIG. 11A, wherein the control element 314 includes a valve 380, the computer system 310 can control a degree of which the valve 380 is opened or closed to modulate the vacuum level and/or aspiration force. For example, in some embodiments, the valve 380 may have an open position and a closed position. In other embodiments, the valve 380 may also have partially open positions where the degree of which the valve is opened (i.e., allowing flow through) can be modulated. Being able to modulate the valve 380 position to control vacuum may be useful to mitigate vessel wall collapse which may happen when excessive vacuum pressure is applied. Further, the computer system 310 can be configured such that the valve 380 will not exceed a pre-determined maximum degree of openness. In some embodiments, the maximum degree of openness may allow about 20%-80% of the maximum flow rate through the catheter 302, where the maximum flow rate is the flow rate when the valve 380 is completely opened. In other embodiments, the maximum degree of openness may allow only about 50% of the maximum flow rate through the catheter 302. The computer system 310 may be preconfigured to have a maximum allowed degree of openness for different vessel types. That is the degree of openness of the valve in an atherectomy procedure may differ from that in a thrombectomy procedure. Because the vessel wall of arteries is thicker and stronger than that of veins, the maximum degree of openness of the valve 380 in an atherectomy procedure may be higher than that of a thrombectomy procedure. The computer system 310 may also be configured to issue an alert when the valve 380 is opened to a degree that has the potential to cause vessel wall collapse, and a threshold to trigger such alert may be different dependent on the type of treatment procedure (i.e., atherectomy or thrombectomy procedure).

In one example, the valve 380 may be a pinch valve having a valve closed position at about 700 steps, a low aspiration mode position at about 685 steps, and a high aspiration mode position at about 670 steps. In another example, the valve 380 may have a closed position at 0% open, a low aspiration mode position at about 10% opened, and a high aspiration mode position at about 25% opened, where the percentage opened is relative to the diameter of the catheter lumen. The valve closed position may be used in a “system ready” state (i.e., when the system is setup for the procedure before starting the treatment), whereas the low aspiration mode position can be used when hunting for UIM, and the high aspiration mode position can be used when the UIM is detected and/or there is an obstruction at the distal tip of the catheter 302.

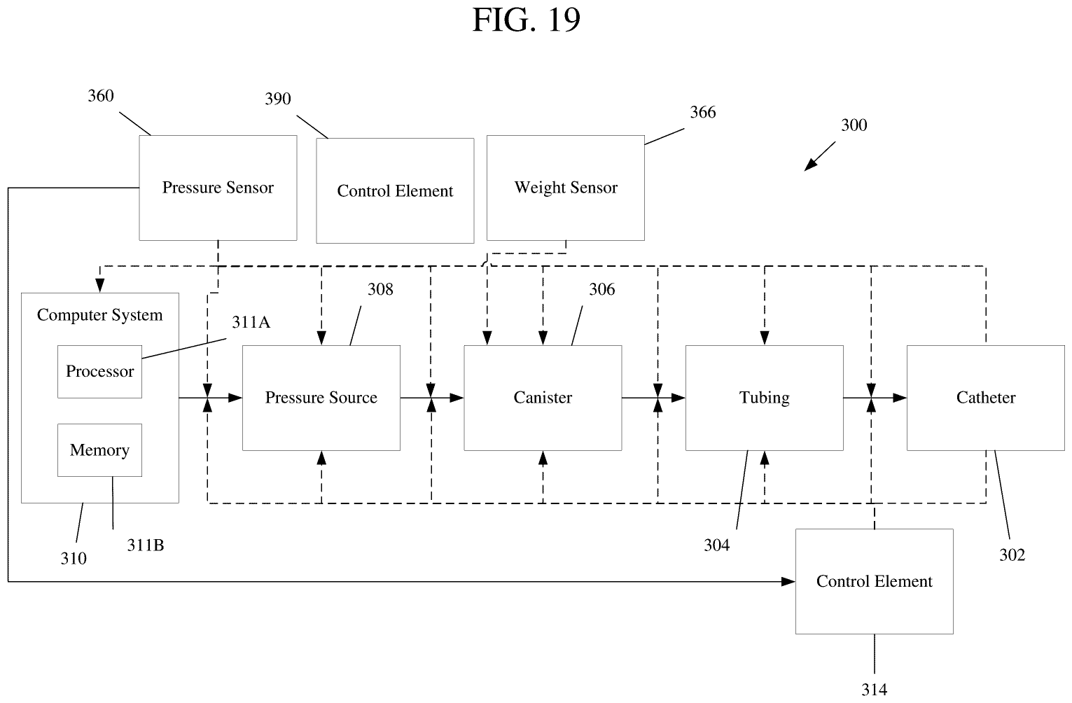

FIG. 19 shows a vascular treatment system 300 according to another embodiment of the disclosure. The vascular treatment system 300 can include a catheter 302 that is configured to be placed into the vasculature of a subject for the treatment and/or removal of UIM, such as the embodiments that are described above in connection with FIGS. 1A-2. The vascular treatment system 300 can further include a canister 306 (or “waste container”) coupled to the catheter 302 via tubing 304. The canister 306 can receive and store the blood and/or UIM that is removed from the subject during treatment for subsequent disposal. The catheter 302 can further be operably coupled to a pressure source 308 (e.g., a pump) that is configured to generate the suction 224 through the catheter 302 for removal and aspiration of the UIM. Further, the vascular treatment system 300 can include a computer system 310 for monitoring the status of the treatment procedure and controlling the operation of the other components of the system 300. The computer system 310 can include a processor 311A coupled to a memory 311B such that the processor 311A can execute instructions stored in the memory 311B to perform various functions embodied by the instructions. In some embodiments, the computer system 310 can provide a graphical user interface (GUI) that allows users to input various parameters, including a target aspiration flow rate, vacuum pressure levels, target energy levels, and so on. Accordingly, the vascular treatment system 300 can be utilized to aspirate UIM in either native or stented vasculature for removal of the UIM from the subject.

FIG. 19 illustrates an embodiment of the vascular treatment system 300 that includes a pressure sensor 362 in combination with a weight sensor 366. In alternative embodiments, the vascular treatment system 300 could include a differential pressure sensor 363 or a pressure sensor assembly 364 in lieu of or in addition to the pressure sensor 362. As described above and indicated in FIG. 19, the pressure sensor 362 could be coupled to a variety of different components of the vascular treatment system 300 and/or be other configured to sense the internal vacuum pressure between a variety of different components of the vascular treatment system 300.

As discussed above, the one or more sensors 312 can be operably coupled to a control element 314 that is configured to modulate the vacuum pressure generated through the catheter 302. Embodiments of the vascular treatment system 300 can include a variety of different control elements 314 that can be configured to control the computer system 310, the pressure source 308, the canister 306, the tubing 304, the catheter 302, or any of the connections between the aforementioned components to modulate the vacuum pressure at the catheter tip for the vascular treatment system 300.

As shown in FIG. 19, the vascular treatment system 300 according to this embodiment also includes a control element 390 for controlling a positive pressure being introduced to the system 300. In one example, the control element 390 may be a solenoid valve for introducing an air leak in the system 300 for reducing a vacuum level. However, in other embodiments, the control element 390 may include different valves, nozzles or gates configured to permit air inflow therethrough. This embodiment allows the vascular treatment system 300 to be used in both the arterial and venous settings. For example, the vessel walls of veins are thinner and weaker than the vessel walls of arteries. Therefore, in order to use the same pressure source 308 for both atherectomy thrombectomy procedures, the vacuum level may need to be adjusted dependent on type of procedure to avoid damage to the vessel wall. The control element 390 can introduce a positive pressure into the system 300 to reduce the vacuum level within the system 300 for thrombectomy procedures.

In one example, the pressure source 310 can be configured to provide a default vacuum pressure of 40 Torr. The control element 390 can be used to introduce a positive pressure air leak to bring the vacuum to approximately 100 Torr for atherectomy procedures. In one example, for atherectomy procedures, the control element 390 can be modulated to introduce a positive pressure air leak to bring the vacuum to a first vacuum level to be used when hunting for UIM (i.e., a low aspiration mode) and a second vacuum level when a UIM or obstruction is detected (i.e., a high aspiration mode), where the second vacuum level is a stronger vacuum than the first vacuum level. In one example, for thrombectomy procedures, the control element 390 can be modulated to introduce a positive pressure air leak to bring the vacuum level to approximately 40-60 Torr when hunting for UIM (i.e., a low aspiration mode) and to approximately 15-45 Torr when UIM or an obstruction is detected (i.e., a high vacuum mode). In another example for thrombectomy procedures, the control element 390 can be modulated to introduce a positive pressure air leak to bring the vacuum level to approximately 50 Torr when hunting for UIM and approximately 20 Torr when UIM or an obstruction is detected. That is, the control element 390 can introduce more or less positive pressure to modulate the vacuum level dependent on the type of procedure and the system state. In other embodiments, control element 390 is not necessary and a pressure source that is configured to adjust the default vacuum level directly can be used.

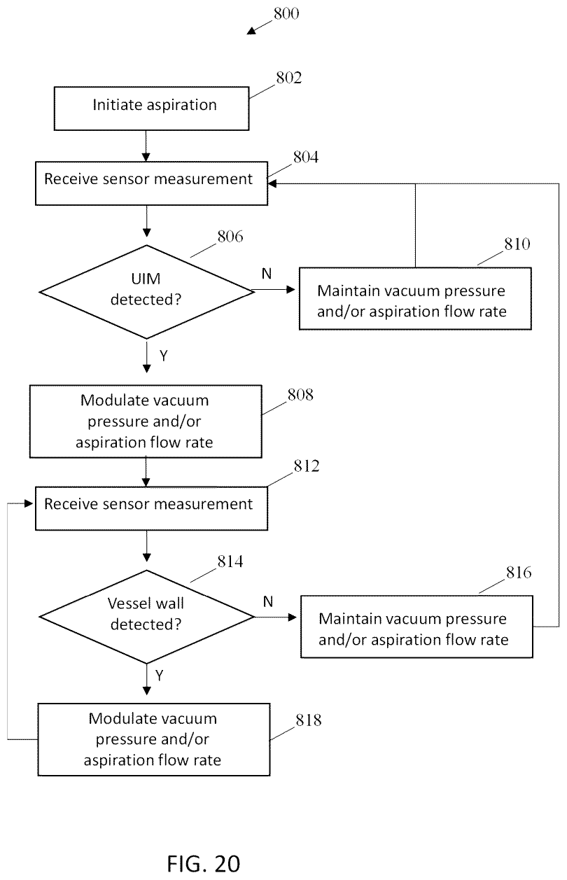

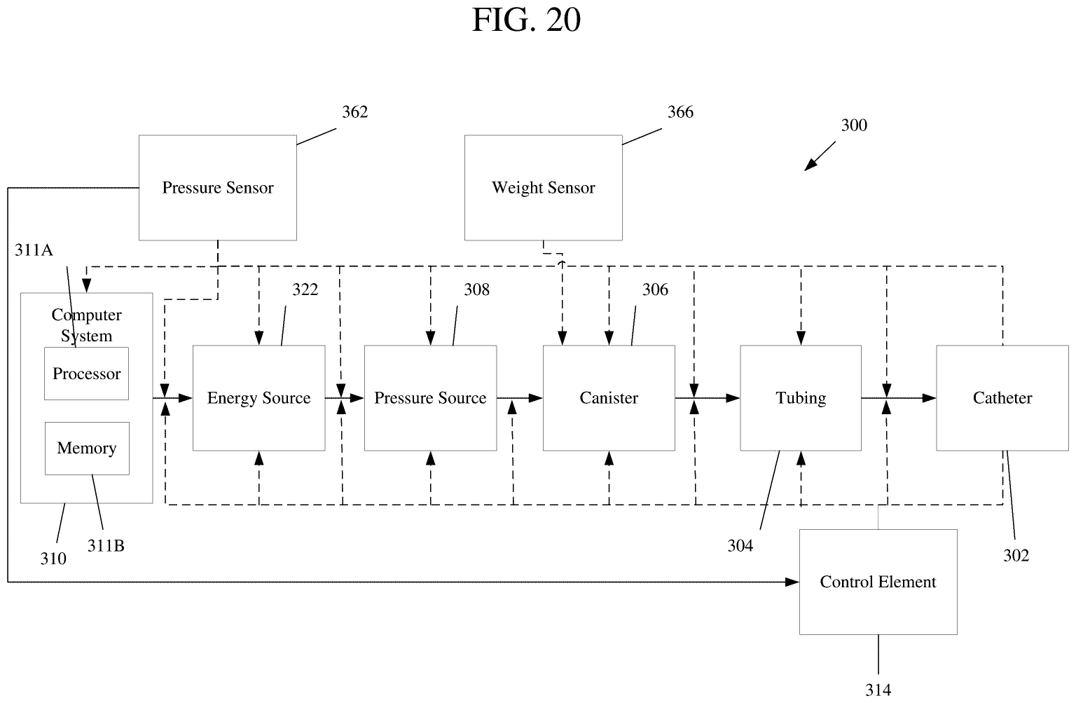

Referring now to FIG. 20, there is shown one embodiment of a process 800 for modulating aspiration flow based on whether a UIM is detected and/or a vessel wall is detected. Accordingly, the computer system 310 executing the process 800 can cause the vascular treatment system 300 to initiate aspiration 802 through the catheter 302 (e.g., by activating the pressure source 308). Aspiration is initiated at a first aspiration flow rate. For example, when using a pressure source 308 capable of applying different vacuum levels (such as by controller 386 of FIG. 14), the pressure source 308 may be activated at a first vacuum level or low vacuum level to cause a first aspiration flow rate or low aspiration flow rate. When using a pressure source having only one vacuum level, the pressure source 308 may be initiated, and the control element 314 may be in a first position to control the aspiration flow rate generated through the catheter 302 thereby generating the first aspiration flow rate.

During aspiration, the computer system 310 can receive a sensor measurement 804 from one or more of the sensors 312. The received sensor measurement could include, for example, direct (e.g., via a flow sensor 360 or pressure sensor 362) and/or indirect (e.g., via a canister weight sensor 366 or other sensors disclosed herein) measurements of the aspiration flow rate. In this embodiment, the computer system 310 can determine whether an UIM is present 806. The computer system 310 could determine that an UIM is present either directly (e.g., by UIM being detected by an optical sensor) or indirectly (e.g., based on a drop in the pressure differential across one or more components of the vascular treatment system 300 or the sensed aspiration flow rate dropping below a threshold) via one or more sensors 312, as described throughout the present disclosure. If the computer system 310 identifies the presence of an UIM, the computer system 310 can modulate the vacuum pressure and/or aspiration flow rate 808. For example, the computer system 310 can modulate the vacuum pressure to a second vacuum level, where the second vacuum level is a higher vacuum level than the first vacuum level (i.e., applying a stronger vacuum level). Additionally or alternatively, the computer system 310 can modulate the aspiration flow rate to a second aspiration flow rate, where the second aspiration flow rate is higher than the first aspiration flow rate (i.e., generating faster flow). The computer system 310 can modulate the vacuum pressure and/or aspiration flow rate using the control element 314 accordingly (e.g., increasing the vacuum level and/or flow aspiration rate), or by adjusting the level of the vacuum output at the pressure source 308. Conversely, if no UIM is detected, the computer system 310 can maintain the vacuum pressure and/or flow aspiration rate 810 at the first vacuum level and/or first aspiration flow rate, receive a sensor measurement 804 and determine whether UIM is detected 806. This allows the system 300 to continue to hunt for UIM without excessive blood loss that may occur when high vacuum levels and/or aspiration flow rates are used continuously, unnecessarily and/or prematurely.

Following modulation of the vacuum pressure 808, the computer system 310 can receive another sensor measurement 812 and determine whether a vessel wall is detected 814. The computer system 310 can determine whether a vessel wall is detected based on a flow rate profile determined by the sensor measurement. For example, when the distal tip of the catheter 302 engages with UIM, the flow rate drops to almost 0 ml/min almost immediately. When the distal tip of the catheter 302 engages a vessel wall, the flow rate decreases slowly until reaching 0 ml/min. Therefore, how quickly the sensor measures the drop in flow rate determines whether the UIM or vessel wall is engaged by the distal tip of the catheter 302.

When the vessel wall is not detected, the computer system 310 can maintain the vacuum pressure and/or aspiration flow rate 816 at the second vacuum level and/or second aspiration flow rate. The computer system 310 may then continue to receive sensor measurements 804 and detect the UIM 806 until the UIM is aspirated. When the vessel wall is detected, the computer system 310 can modulate the vacuum pressure and/or aspiration flow rate 818. For example, the computer system 310 can periodically reduce the vacuum level and/or aspiration flow rate to the first vacuum level or first aspiration flow rate. That is, the computer system 310 can switch back and forth between the first vacuum level/first aspiration flow rate (i.e., low vacuum or low flow) and second vacuum level/second aspiration flow rate (i.e., high vacuum or high flow). Doing so mitigates risks associated with damaging the vessel wall due to application of high vacuum on the vessel wall for long periods of time. The computer system 310 may administer a programmed vacuum modulation scheme where high vacuum and/or high flow is applied for a set period of time followed by low vacuum and/or low flow being applied for a set period of time. In one example, the high vacuum level and/or high aspiration flow rate may be applied for approximately 3 seconds while the low vacuum level and/or low aspiration flow rate may be applied for approximately 0.5 seconds. This cycle may be repeated for the entire duration that the computer system 310 is in a high aspiration mode or may be triggered only when the vessel wall is detected. In other embodiments, the computer system 310 may repeat this cycle for a predetermined total amount of time. The computer system 310 may modulate the vacuum level and/or aspiration flow rate by adjusting the control element 314 accordingly or by adjusting the vacuum output of the pressure source 308. Where the control element 314 is a valve, this can include modulating the degree of openness of the valve to control the flow rate within the catheter 302. In some embodiments, modulating the control element 314 can include modulating the degree of openness of the control element 314 without completely closing the control element 314 or stopping application of the vacuum at the distal tip of the catheter 302. In other embodiments, modulating the control element 314 can include modulating the degree of openness of the control element 314 including completely closing the control element 314 to stop application of the vacuum at the distal tip of the catheter 302. Modulating the vacuum level in this way allows for the vessel wall to reinflate during periods of low vacuum level and/or low aspiration flow, and therefore, mitigates the risk of damaging the vessel wall. The computer system 310 can iteratively receive sensor measurements, determine UIM states, and modulate the vacuum pressure and/or aspiration flow rates accordingly until all of the UIM is aspirated and/or a clinician determines the treatment procedure to be complete.

### Smart Aspiration and Laser Activation for Vascular Treatment Systems

In addition to controlling aspiration flow rates and vacuum pressures through the catheter 302 of the system 300, aspects of the disclosure also contemplate controlling energy radiation emitted by the optical fibers 104, 222 positioned at the distal end of the catheter 302. For example, it may be desirable to only activate the energy source when a UIM is detected and unable to be removed via aspiration alone, or when there is an obstruction identified at the distal tip of the catheter 302. Therefore, this embodiment allows for longer treatments by limiting the amount of time that the energy source is activated and/or applied within the vessel. Additionally, such an embodiment provides safety to the vessel wall since lasing is to take place only when the system detects a UIM, and further in some cases, only when there is a clog in the system or the UIM obstructs the catheter.

FIG. 21 shows a vascular treatment system 300 according to another embodiment of the disclosure having an energy source. While shown in described relative to this embodiment, it is to be understood that the energy source 322 and the energy radiation activation processes can apply to any of the embodiments discussed herein such as any of the embodiments of FIGS. 3B-15. The vascular treatment system 300 can include a catheter 302 that is configured to be placed into the vasculature of a subject for the treatment and/or removal of UIM, such as the embodiments that are described above in connection with FIGS. 1A-2. The vascular treatment system 300 can further include a canister 306 (or “waste container”) coupled to the catheter 302 via tubing 304. The canister 306 can receive and store the blood and/or UIM that is removed from the subject during treatment for subsequent disposal. The catheter 302 can further be operably coupled to a pressure source 308 (e.g., a pump) that is configured to generate the suction 224 through the catheter 302 for removal and aspiration of the UIM. The catheter 302 can further be operably coupled to an energy source 322 (e.g., a laser system, optical box, etc.). For example, as described elsewhere herein, the energy source 322 can be operatively coupled to optical fibers that extend the length of the catheter 302 such that the optical fibers emit energy radiation about the distal tip of the catheter 302 for application of the energy radiation to the UIM as described elsewhere herein. Applying energy radiation to the UIM helps break down the UIM for easier aspiration. Further, the vascular treatment system 300 can include a computer system 310 for monitoring the status of the treatment procedure and controlling the operation of the other components of the system 300. The computer system 310 can include a processor 311A coupled to a memory 311B such that the processor 311A can execute instructions stored in the memory 311B to perform various functions embodied by the instructions. In some embodiments, the computer system 310 can provide a graphical user interface (GUI) that allows users to input various parameters, including a target aspiration flow rate, vacuum pressure levels, target energy levels, energy radiation levels, energy radiation times, and so on. Accordingly, the vascular treatment system 300 can be utilized to aspirate UIM in either native or stented vasculature for removal of the UIM from the subject.

FIG. 21 illustrates an embodiment of the vascular treatment system 300 that includes a pressure sensor 362 in combination with a weight sensor 366. In alternative embodiments, the vascular treatment system 300 could include a differential pressure sensor 363 or a pressure sensor assembly 364 in lieu of or in addition to the pressure sensor 362. As described above and indicated in FIG. 19, the pressure sensor 362 could be coupled to a variety of different components of the vascular treatment system 300 and/or be other configured to sense the internal vacuum pressure between a variety of different components of the vascular treatment system 300.

As discussed above, the one or more sensors 312 can be operably coupled to a control element 314 that is configured to modulate the vacuum pressure and/or aspiration flow rate generated through the catheter 302. Embodiments of the vascular treatment system 300 can include a variety of different control elements 314 that can be configured to control the computer system 310, the pressure source 308, the energy source 322, the canister 306, the tubing 304, the catheter 302, or any of the connections between the aforementioned components to modulate the vacuum pressure, aspiration flow rate, and/or energy radiation at the catheter tip for the vascular treatment system 300.

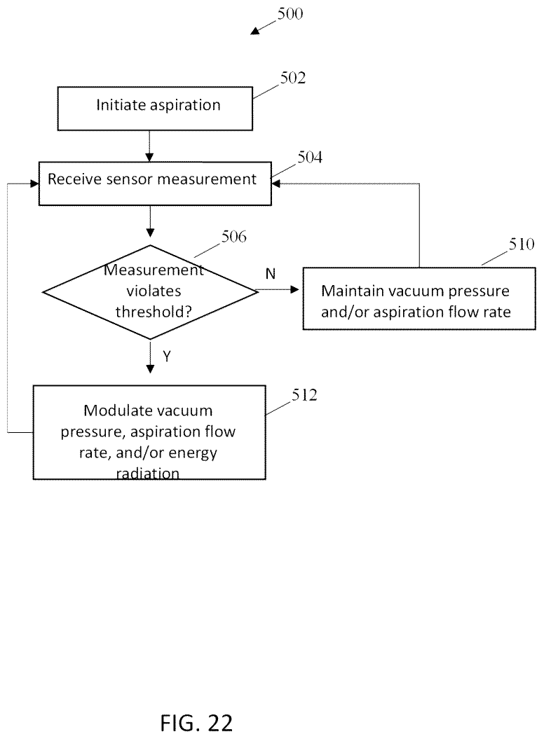

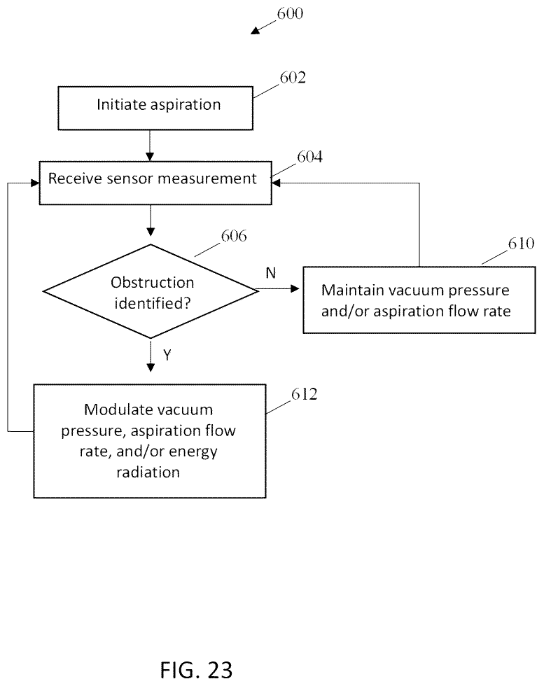

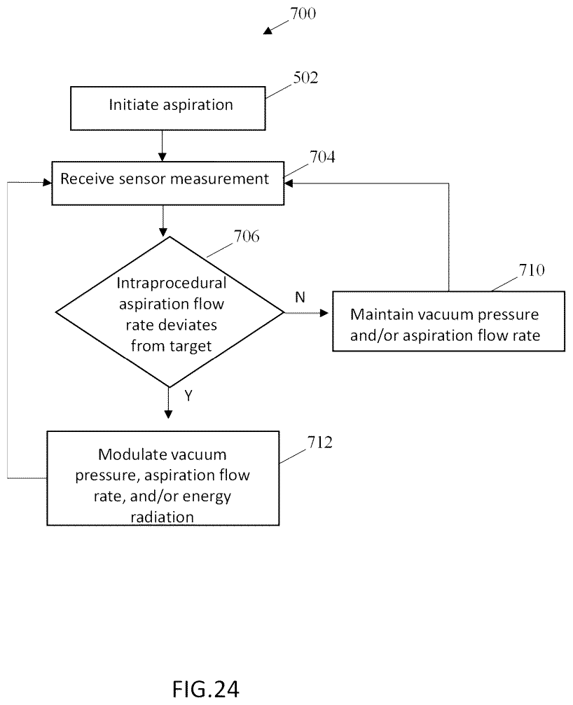

Referring now to FIG. 22, there is shown an alternative embodiment of the process 500 of FIG. 16. In this embodiment, the process is substantially the same as that of FIG. 16 except that instead of modulating the vacuum pressure, and/or aspiration flow rate 508 when the measurement violates the threshold 506, the computer system may modulate the vacuum pressure, aspiration flow rate, and/or the energy radiation 512 at the distal tip of the catheter. Similarly, FIG. 23 shows an alternative embodiment of the process 600 of FIG. 17. In this embodiment, the process is substantially the same as that of FIG. 17 except that instead of modulating the vacuum pressure and/or aspiration flow rate 608 when the obstruction is identified 606, the computer system may modulate the vacuum pressure, aspiration flow rate, and/or the energy radiation 612 at the distal tip of the catheter. Further, FIG. 24 shows an alternative embodiment of the process 700 of FIG. 18. In this embodiment, the process is substantially the same as that of FIG. 18 except that instead of modulating the vacuum pressure and/or aspiration flow rate 708 when the intraprocedural aspiration flow rate deviates from a target 706, the computer system may modulate the vacuum pressure, aspiration flow rate, and/or the energy radiation 712 at the distal tip of the catheter 302.

In any of the embodiments of FIGS. 22-24, aspiration can be initiated at a first vacuum level and/or first aspiration flow rate, and the modulating of the vacuum pressure and/or aspiration flow rate may include modulating the vacuum pressure to a second vacuum level, greater than the first vacuum level and/or modulating the aspiration flow rate to a second aspiration flow rate, greater than the first aspiration flow rate. That is, where the measurement violates the threshold 506 (FIG. 22), the obstruction is identified 606 (FIG. 23), and/or the intraprocedural aspiration flow rate deviates from a target 706 (FIG. 24), the vacuum pressure and/or aspiration flow rate could be increased to initiate a stronger vacuum and/or faster aspiration flow rate applied at the distal tip of the catheter 302.

In any of the embodiments of FIGS. 22-24, the modulating of the vacuum pressure, aspiration flow rate, and/or energy radiation may include modulating one or both of the vacuum pressure, aspiration flow rate, and energy radiation. In one example, the vacuum pressure and/or aspiration flow rate may first be modulated to the second/higher vacuum level and/or second/higher aspiration flow rate, and then another sensor measurement may be received. The computer system 310 can again determine whether the measurement violates a threshold (FIG. 22), identifies an obstruction (FIG. 23), or an intraprocedural aspiration flow rate deviates from the target (FIG. 24). If the computer system 310 finds that any of these conditions are met, the computer system 310 may then modulate the energy source 322 to generate an energy radiation at the distal tip of the catheter 302 to break up the UIM and aid in aspiration thereof. If the computer system 310 finds that none of these conditions are met, the computer system 310 can modulate the vacuum pressure to the first vacuum level thereby reducing the vacuum level generated at the distal tip of the catheter 302 and/or modulate the aspiration flow rate to the first aspiration flow rate thereby reducing the aspiration flow rate generated at the distal tip of the catheter to prevent excessive blood loss.

In some embodiments, a determination that the measurement violates a threshold may correspond to the tip of the catheter being proximate the UIM in the vasculature, an obstruction at the tip of the catheter 302, or an obstruction in the system 300 (e.g., in the tubing 304 or catheter 302). In some embodiments, the threshold comprises a target aspiration flow rate and the modulation of the vacuum pressure and/or energy radiation causes an aspiration flow rate of the fluid and UIM to align with the target aspiration flow rate.

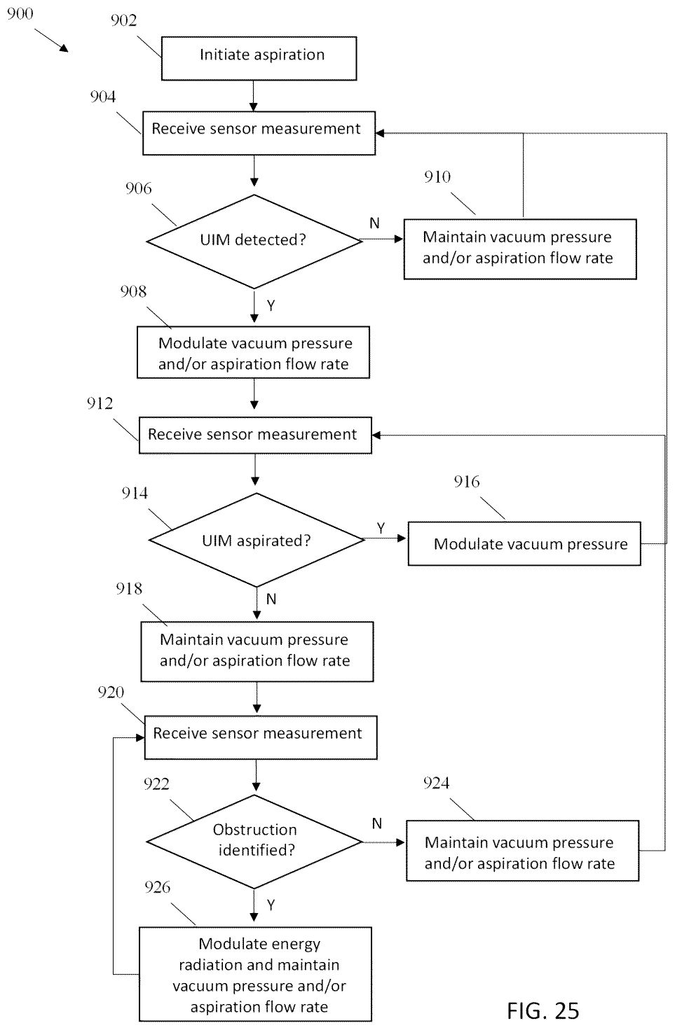

Referring now to FIG. 25, there is shown one embodiment of a process 900 for modulating aspiration flow and energy radiation. Accordingly, the computer system 310 executing the process 900 can cause the vascular treatment system 300 to initiate aspiration 902 through the catheter 302 (e.g., by activating the pressure source 308). Aspiration is initiated at a first vacuum level and/or a first aspiration flow rate. For example, when using a pressure source 308 capable of applying different vacuum levels, the pressure source 308 may be activated at a first vacuum level. When using a pressure source 308 having only one vacuum level, the pressure source 308 may be initiated, and the control element 314 may be in a first position to control the vacuum pressure and/or aspiration flow rate generated through the catheter 302 thereby applying the first vacuum level and/or aspiration flow rate. At this time, the energy source 322 may be deactivated or turned off.

During aspiration, the computer system 310 can receive a sensor measurement 904 from one or more of the sensors 312. The received sensor measurement could include, for example, direct (e.g., via a flow sensor 360 or pressure sensor 362) or indirect (e.g., via a canister weight sensor 366 or other sensors discussed herein) measurements of the aspiration flow rate. In this embodiment, the computer system 310 can determine whether an UIM is present 906. The computer system 310 could determine that an UIM is present either directly (e.g., by UIM being detected by an optical sensor) or indirectly (e.g., based on a drop in the pressure differential across one or more components of the vascular treatment system 300 or the sensed aspiration flow rate dropping below a threshold) via one or more sensors 312, as described throughout the present disclosure. Similar to the aforementioned embodiment, if the computer system 310 identifies the presence of an UIM, the computer system 310 can modulate the vacuum pressure and/or aspiration flow rate 908. For example, the computer system 310 can modulate the vacuum pressure to a second vacuum level, where the second vacuum level is a higher vacuum level than the first vacuum level (i.e., applying a stronger vacuum level). Additionally or alternatively, the computer system 310 can modulate the aspiration flow rate to a second aspiration flow rate, where the second aspiration flow rate is faster than the first aspiration flow rate. The computer system 310 can modulate the vacuum pressure and/or aspiration flow rate using the control element 314 accordingly (e.g., increasing the vacuum level and/or aspiration flow rate), or by adjusting the level of the vacuum output at the pressure source 308. As discussed elsewhere herein, applying the increased vacuum level and/or aspiration flow rate may include a programmed vacuum modulation scheme where high vacuum and/or high aspiration flow is applied for a set period of time followed by low vacuum and/or low aspiration flow being applied for a set period of time. Conversely, if no UIM is detected, the computer system 310 can maintain the vacuum pressure and/or aspiration flow rate at the first vacuum level and/or first aspiration flow rate, and again receive a sensor measurement 904 and determine whether UIM is detected 906. This allows the system 300 to continue to hunt for UIM without excessive blood loss that may occur when high vacuum levels and/or high aspiration flow rates are used unnecessarily.

Following modulation of the vacuum pressure and/or aspiration flow rate 908, the computer system 310 can receive another sensor measurement 912 and determine whether the UIM has been aspirated 914. The computer system 310 can determine whether the UIM has been aspirated either directly (e.g., by UIM no longer being detected by an optical sensor) or indirectly (e.g., based on an increase in the pressure differential across one or more components of the vascular treatment system 300 or the sensed aspiration flow rate increasing above a threshold). If the computer system 310 determines that the UIM was aspirated, the computer system 310 can modulate the vacuum pressure and/or aspiration flow rate 916. For example, the computer system 310 can modulate the vacuum pressure and/or aspiration flow rate to the first vacuum level and/or first aspiration flow rate or some vacuum level and/or aspiration flow rate lower than the second vacuum level and/or second aspiration flow rate used to aspirate the UIM. The computer system 310 can modulate the vacuum pressure and/or aspiration flow rate using the control element 314 accordingly (e.g., decreasing the vacuum level and/or aspiration flow rate), or by adjusting the level of the vacuum output at the pressure source 308. Reducing the vacuum pressure and/or aspiration flow rate at this stage reduces excessive blood loss. At this time, the clinician can determine whether the treatment procedure is complete.

If the computer system 310 determines that the UIM was not aspirated at 914, the computer system 310 can maintain the vacuum pressure and/or aspiration flow rate 918 at the second vacuum level and/or second aspiration flow rate until the UIM is aspirated. However, in other embodiments, the computer system 310 could again increase the vacuum level to another vacuum level greater than the second vacuum level (e.g., thereby applying a stronger vacuum) and/or increase the aspiration flow rate to another aspiration flow rate greater than the second aspiration flow rate to aspirate the UIM. Following 918, the computer system 310 can receive another sensor measurement 920 to determine whether an obstruction is identified 922 at the distal tip of the catheter 302. The computer system 310 can determine whether there is an obstruction either directly (e.g., by UIM being detected by an optical sensor) or indirectly (e.g., based on a drop in the pressure differential across one or more components of the vascular treatment system 300 or the sensed aspiration flow rate dropping below a threshold) via one or more sensors 312, as described throughout the present disclosure. If no obstruction has been identified, the computer system 310 can maintain vacuum pressure and/or aspiration flow rate 924 to continue to aspirate UIM and return to 912 to receive a sensor measurement and 914 to determine whether UIM was aspirated. If an obstruction has been identified, the computer system 310 can modulate the energy radiation 926. For example, the computer system 310 can activate or turn on the energy source 322 to generate energy radiation on the distal tip of the catheter 302. The energy radiation can be configured to break up the UIM so that the UIM can be more easily aspirated via the catheter 302. This embodiment activates the energy source 322 only when UIM obstructs the distal tip of the catheter 302, thereby preventing unwanted damage to the vessel wall due to excessive energy radiation. At this time, the vacuum pressure and/or the aspiration flow rate can be maintained until the UIM is aspirated. However, in other embodiments, the computer system 310 could again increase the vacuum level and/or aspiration flow rate to another vacuum level greater than the second vacuum level (e.g., thereby applying a stronger vacuum and/or faster aspiration flow) to aspirate the UIM. In further embodiments, the computer system 310 can lower the vacuum level to the first vacuum level (e.g., thereby applying a weaker vacuum) and/or lower the aspiration flow rate to the first aspiration flow rate (e.g., generating a lower aspiration flow) which could be used due to the ease at which aspiration of the UIM may take place following breaking up of the UIM due to application of energy radiation. Following 926, the computer system 310 can again receive a sensor measurement 920 and determine whether the obstruction has been cleared 922. The computer system 310 can iteratively receive sensor measurements, determine UIM states, and modulate the vacuum pressure, aspiration flow rate, and/or energy radiation accordingly until all of the UIM is aspirated and/or a clinician determines the treatment procedure to be complete.

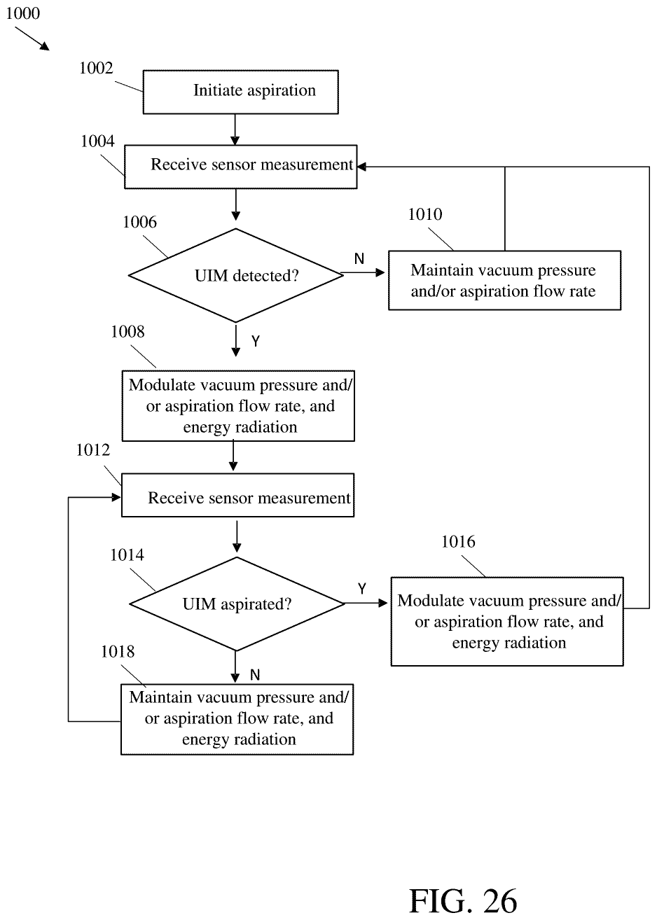

Referring now to FIG. 26, there is shown one embodiment of a process 1000 for modulating aspiration flow and energy radiation. Accordingly, the computer system 310 executing the process 1000 can cause the vascular treatment system 300 to initiate aspiration 1002 through the catheter 302 (e.g., by activating the pressure source 308). Aspiration is initiated at a first vacuum level and/or a first aspiration flow rate. For example, when using a pressure source 308 capable of applying different vacuum levels, the pressure source 308 may be activated at a first vacuum level. When using a pressure source 308 having only one vacuum level, the pressure source 308 may be initiated, and the control element 314 may be in a first position to control the vacuum pressure and/or aspiration flow rate generated through the catheter 302 thereby applying the first vacuum level and/or first aspiration flow rate. At this time, the energy source 322 may be deactivated or turned off.

During aspiration, the computer system 310 can receive a sensor measurement 1004 from one or more of the sensors 312. The received sensor measurement could include, for example, direct (e.g., via a flow sensor 360 or pressure sensor 362) or indirect (e.g., via a canister weight sensor 366) measurements of the aspiration flow rate. In this embodiment, the computer system 310 can determine whether an UIM is present 1006. The computer system 310 could determine 1006 that an UIM is present either directly (e.g., by UIM being detected by an optical sensor) or indirectly (e.g., based on a drop in the pressure differential across one or more components of the vascular treatment system 300 or the sensed aspiration flow rate dropping below a threshold) via one or more sensors 312, as described throughout the present disclosure. Similar to the aforementioned embodiment, if the computer system 310 identifies the presence of an UIM, the computer system 310 can modulate the vacuum pressure and/or aspiration flow rate 1008. For example, the computer system 310 can modulate the vacuum pressure to a second vacuum level, where the second vacuum level is a higher vacuum level (i.e., applying a stronger vacuum level) than the first vacuum level. Additionally or alternatively, the computer system 310 can modulate the aspiration flow rate to a second aspiration flow rate, where the second aspiration flow rate is faster than the first aspiration flow rate. The computer system 310 can modulate the vacuum pressure and/or aspiration flow rate using the control element 314 accordingly (e.g., increasing the vacuum level), or by adjusting the level of the vacuum output at the pressure source 308. As discussed elsewhere herein, applying the increased vacuum level and/or aspiration flow rate may include a programmed vacuum modulation scheme where high vacuum and/or high aspiration flow is applied for a set period of time followed by low vacuum and/or low aspiration flow being applied for a set period of time. However, in this embodiment, process 1000 further includes modulating energy radiation at 1008. For example, the energy source 322 can be activated or turned on to generate energy radiation at the distal tip of the catheter 302. The energy radiation can be configured to break up the UIM so that the UIM can be more easily aspirated via the catheter 302. This embodiment activates the energy source 322 when UIM is detected thereby preventing unwanted damage to the vessel wall due to excessive energy radiation.

Conversely, if no UIM is detected, the computer system 310 can maintain the vacuum pressure and/or aspiration flow rate 1010 at the first vacuum level and/or first aspiration flow rate, and again receive a sensor measurement 1004 and determine whether UIM is detected 1006. This allows the system 300 to continue to hunt for UIM without excessive blood loss that may occur when high vacuum levels and/or aspiration rates are used unnecessarily.

Following modulation of the vacuum pressure and/or aspiration flow rate 1008, the computer system 310 can receive another sensor measurement 1012 and determine whether the UIM has been aspirated 1014. The computer system 1014 can determine whether the UIM has been aspirated either directly (e.g., by UIM no longer being detected by an optical sensor) or indirectly (e.g., based on an increase in the pressure differential across one or more components of the vascular treatment system 300 or the sensed aspiration flow rate increasing above a threshold). If the computer system 310 determines that the UIM was aspirated, the computer system 310 can modulate the vacuum pressure and/or aspiration flow rate 1016. For example, the computer system 310 can modulate the vacuum pressure and/or aspiration flow rate to the first vacuum level and/or first aspiration flow rate or some vacuum level or aspiration flow rate lower than the second vacuum level and/or second aspiration flow rate used to aspirate the UIM. The computer system 310 can modulate the vacuum pressure and/or aspiration flow rate using the control element 314 accordingly (e.g., decreasing the vacuum level and/or aspiration flow rate), or by adjusting the level of the vacuum output at the pressure source 308. Reducing the vacuum pressure and/or aspiration flow rate at this stage reduces excessive blood loss. At this time the clinician can determine whether the treatment procedure is complete.

If the computer system 310 determines that the UIM was not aspirated at 1014, the computer system 310 can maintain the vacuum pressure and/or aspiration flow rate 1018 at the second vacuum level and/or second aspiration flow rate, and maintain the energy radiation until the UIM is aspirated. However, in other embodiments, the computer system 310 could again increase the vacuum level and/or aspiration flow rate to another vacuum level and/or aspiration flow rate greater than the second vacuum level (e.g., thereby applying a stronger vacuum) and/or second aspiration flow rate (e.g., thereby generating a faster flow) to aspirate the UIM. Additionally or alternatively, the computer system 310 can adjust a parameter of the energy radiation outputted by the energy source 322 and/or catheter 302 distal tip to modulate the energy radiation. For example, one or more of a wavelength, photon energy, fluence, pulse paradigm, etc. can be adjusted at the energy source 322. Following 1014, the computer system 310 can again receive a sensor measurement 1012 and determine whether the UIM has been aspirated 1014. The computer system 310 can iteratively receive sensor measurements, determine UIM states, and modulate the vacuum pressure, aspiration flow rate, and/or energy radiation accordingly until all of the UIM is aspirated and/or a clinician determines the treatment procedure to be complete.

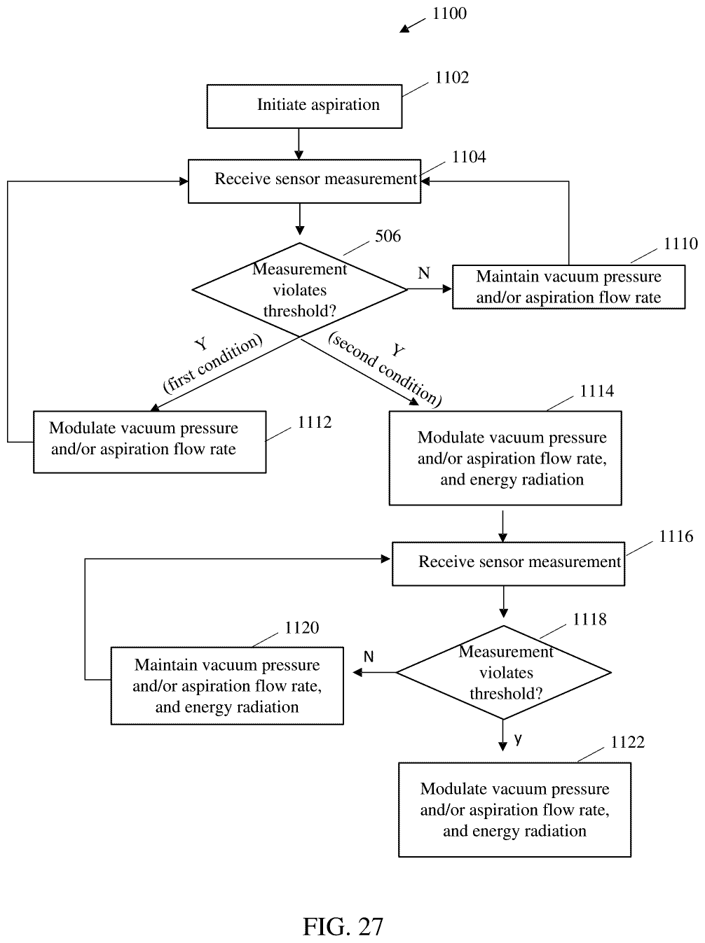

Turning now to FIG. 27, there is shown one embodiment of a process 1000 for modulating aspiration flow and energy radiation. Accordingly, the computer system 310 executing the process 1000 can cause the vascular treatment system 300 to initiate aspiration 1002 through the catheter 302 (e.g., by activating the pressure source 308). In this process 1100, the process includes initiating aspiration 1102 by activating a pump to initiate a first vacuum pressure and/or first aspiration flow rate to be generated through the catheter 302 to aspirate a fluid and UIM. A first measurement of a parameter associated with a sensor can be received 1104 and the computer system 310 can determine whether the first measurement violates a threshold associated with the parameter thereby determining a state of the system 1106. Where the first measurement does not violate the threshold, the first vacuum pressure and/or first aspiration flow rate is maintained, and the state of the system is determined to be a free flow state.

Where the first measurement is determined to violate the threshold according to a first condition, the state of the system is determined to be in a UIM detected state and the vacuum pressure and/or aspiration flow rate is modulated 1112. This can include modulating the pressure source 308 to generate a second vacuum pressure through the catheter 302. The second vacuum pressure is a stronger vacuum pressure than the first vacuum pressure. As discussed elsewhere herein, applying the increased vacuum level may include a programmed vacuum modulation scheme where high vacuum is applied for a set period of time followed by low vacuum being applied for a set period of time. Additionally or alternatively, this can include modulating a control element 314 to generate the second vacuum pressure and/or a second aspiration flow rate through the catheter 302. The second aspiration flow rate is a faster flow rate than the first aspiration flow rate. As discussed elsewhere herein, applying the increased aspiration flow rate may include a programmed vacuum modulation scheme where high aspiration is applied for a set period of time followed by low aspiration being applied for a set period of time. The computer system 310 can again receive sensor measurements 1104, determine whether the measurement violates the threshold 506, and modulate the vacuum pressure and/or aspiration flow rate accordingly until the UIM is aspirated.

Where the first measurement is determined to violate the threshold according to the second condition, the state of the system is determined to be in an obstructed state and the pressure source and/or aspiration flow rate 308 is modulated to generate the second vacuum pressure and/or second aspiration flow rate through the catheter 302. Additionally, the energy source 322 is activated to generate an energy radiation 1114 at the distal end of the catheter 302. The computer system 310 can receive another sensor measurement 1116 and determine whether the measurement violates a threshold 1118. Where the threshold is not violated, the computer system 310 can maintain the vacuum pressure and/or aspiration flow rate, and energy radiation 1120 to continue to break up and aspirate the UIM. Where the threshold is violated according to another condition, the computer system 310 can modulate the vacuum pressure and/or aspiration flow rate, and energy radiation 1122. More specifically, the energy source 322 may be deactivated or turned off. In other embodiments, the energy radiation can be reduced such as by adjusting a parameter. Additionally, the computer system 310 can reduce the vacuum pressure to return the vacuum pressure to the first vacuum level and/or reduce the aspiration flow rate to return the aspiration flow to the first aspiration flow rate.

As indicated in FIGS. 22-27, regardless of whether the computer system 310 modulates or maintains the vacuum pressure, aspiration flow rate, and/or energy radiation at the tip of the catheter 302, the computer system 310 can continue receiving sensor measurements and acting accordingly to control the vacuum pressure, aspiration flow rate, and/or energy radiation at the catheter tip 302 in response to the detection of any UIM or obstructions. According to this embodiment, the vascular treatment system 300 applies a low vacuum and/or aspiration with little to no energy radiation during times when there is no UIM and/or no obstruction detected thereby reducing the likelihood of vessel wall damage and excessive blood loss enabling longer treatment times.

It is to be understood that, while FIG. 21 shows the embodiment of FIG. 10B with the energy source 322 in operative communication with the computer system 310, any of the vascular treatment systems 300 discussed herein also may have the energy source 322 in operative communication with the computer system 310 such that any of the processes described herein can be performed with any of the vascular treatment systems 300 disclosed.

### EXAMPLES

The following examples are simply provided to illustrate potential measurements and responses in some embodiments of the vascular treatment system 300 and should not be understood to be limiting in any way.

The following table shows an example of sensor measurements that trigger the computer system 310 to move between system states in an embodiment where the energy source 322 is turned on or activated only when there is an obstruction detected.

TABLE 5

| | Control Element | Energy Source |
|---|---|---|
| System State | Position | Activation |
| Free flow | Partially closed | Off |
| | ‘Low flow’ | |
| UIM detected | Open | Off |
| | ‘High flow’ | |
| Line obstructed | Open | On |
| | ‘High flow’ | |

In a first example, the system 300 may include both a pressure sensor 362 positioned about a length of the catheter and/or connection tubing and weight sensor 366 disposed on or within the cannister 306 such as that shown in FIG. 10B.

TABLE 6

Sensor output trigger

| | Output of weight | |
|---|---|---|
| Output of vacuum sensor | sensor | Response |
| Stable | Flowrate below 100 | Remain in previous state |
| | g/min | |
| Increase of over 1-20 Torr | Flowrate over 100- | If one of two conditions |
| | 300 g/min | occur, move to ‘free flow’ |
| Drop of over 1-20 Torr | Flowrate under 15 | If one of two conditions |
| | g/min | occur, move to ‘clot |
| | | detected’ |
| Below 100-300 Torr | Below 100-300 | If both conditions occur, |
| | g/min | move to ‘line obstructed’ |

In this example, the catheter 302 may be advanced to a treatment site within a vessel having a UIM. The system 300 may be activated by the user such that the system 300 starts in a “free flow” state. That is, the system 300 may start such that the control element 314 is partially closed to impart low flow through the catheter 302 thereby aspirating at a low flow rate. This minimizes blood loss until the UIM is engaged by the catheter distal tip. Additionally, the energy source 322 is off or not activated in order to minimize damage to the vessel. The system 300 may stay in this state so long as the vacuum pressure remains relatively stable and the flow rate within the cannister 306 remains approximately 15-45 g/min. That is, if the vacuum pressure remains relatively stable and the flow rate within the cannister remains approximately 15-45 g/min, a UIM has not been detected and the system 300 remains in the “free flow” state.

Once the catheter 302 is advanced to the UIM and the catheter distal tip engages the UIM, a drop in vacuum pressure may be observed. That is, the vacuum pressure may experience a decrease of approximately 1-20 Torr due to engagement of the UIM by the catheter distal tip. Additionally, flow within the cannister 306 may be under 15 g/min. As a result, the system 300 may determine that a UIM has been detected and transition to a “UIM detected” state such that the control element 314 opens to impart a high aspiration flow rate through the catheter 302 to aspirate the UIM. In this embodiment, the energy source 322 remains off or not activated to prevent damage to the vessel.

At this stage, the system 300 may remain in the “UIM detected” state if the vacuum remains relatively stable and the computer system 300 is measuring a flow rate of less than approximately 100 g/min (based on reading from the weight sensor). That is, the control element 314 remains open to continue to impart high aspiration flow and the energy source 322 remains off.

However, if the vacuum pressure increases over approximately 1-20 Torr and/or the computer system 310 measures a flow rate of approximately 100-300 g/min within the cannister 306 (based on a reading from the weight sensor), the system 310 may determine that the UIM has been aspirated and the system 300 may move back to the “free flow” state. That is, the control element 314 returns to a partially closed position to impart a low aspiration flow rate to prevent blood loss and the energy source 322 remains off or not activated. If it is determined that the treatment is complete, the system 300 can be turned off and the user can remove the catheter 302 from the vessel.

If the vacuum pressure decreases by approximately 100-300 Torr and the computer system 310 is measuring a flow rate of below 100-300 g/min (based on a reading from the weight sensor 306), the system 310 may determine that the UIM has not been aspirated and therefore, moves to a “line obstructed” state. That is, the control element 314 remains open to continue to impart high aspiration flow and the energy source 322 is turned on or is activated to soften and/or break up the UIM so the UIM can be more easily aspirated. Once the vacuum pressure increases over approximately 1-20 Torr and/or the computer system 310 measures a flowrate of approximately 100-300 g/min within the cannister 306 (based on a reading from the weight sensor), the system 310 may determine that the UIM has been aspirated. As a result, the system 300 may move back to the “free flow” state and the energy source 322 may be shut off and/or not activated and the control element 314 may be partially closed to impart low flow. If it is determined that the treatment is complete, the system 300 can be turned off and the user can remove the catheter 302 from the vessel.

Table 8 shows an example of sensor measurements that trigger the computer system 310 to move between system states in an embodiment where the energy source 322 is turned on or activated when UIM is detected and when there is an obstruction detected. That is, the system 300 may have a control element 314 position and energy source 322 activation based on the following table.

TABLE 7

| System State | Control Element | Energy Source |
|---|---|---|
| | Position | Activation |
| Free flow | Partially closed | Off |
| | ‘Low flow’ | |
| UIM detected | Open | On |
| | ‘High flow’ | |
| Line obstructed | Open | On |
| | ‘High flow’ | |

In one example, the system 300 may include both a pressure sensor 362 positioned about a length of the catheter and/or connection tubing and weight sensor 366 disposed on or within the cannister 306 such as that shown in FIG. 10B.

TABLE 8

| Sensor output trigger | | |
|---|---|---|
| | Output of weight | |
| Output of vacuum sensor | sensor | Response |
| Stable | Flowrate below 100 | Remain in previous |
| | g/min | state |
| Increase of over 1-20 Torr | Flowrate over 100- | If one of two |
| | 300 g/min | conditions occur, |
| | | move to ‘free flow’ |
| Drop of over 1-20 Torr | Flowrate under 15 | If one of two |
| | g/min | conditions occur, |
| | | move to ‘clot |
| | | detected’ |

In this example, the catheter 302 may be advanced to a treatment site within a vessel having a UIM. The system 300 may be activated by the user such that the system 300 starts in a “free flow” state. That is, the system 300 may start such that the control element 314 is partially closed to impart low flow through the catheter 302 thereby aspirating at a low flow rate. This minimizes blood loss until the UIM is engaged by the catheter distal tip. Additionally, the energy source 322 is off or not activated in order to minimize damage to the vessel. The system 300 may stay in this state so long as the vacuum pressure remains relatively stable and the flow rate within the cannister 306 remains below approximately 100 g/min.

Once the catheter 302 is advanced to the UIM and the catheter distal tip engages the UIM, a drop in vacuum pressure may be observed. That is, the vacuum pressure may experience a decrease of approximately 1-20 Torr due to engagement of the UIM by the catheter distal tip. Additionally, flow within the cannister 306 may be under 15 g/min. As a result, the system 300 may transition to a “UIM detected” state such that the computer system 310 opens the control element 314 to impart high flow through the catheter to aspirate the UIM. In this embodiment, the energy source 322 turns on or is activated by the computer system 310 to soften or break up the UIM such that the UIM can be more easily aspirated.

At this stage, the system 300 may remain in the “UIM detected” state if the vacuum remains relatively stable and the computer system 310 is measuring a flow rate of below 100 g/min (based on a reading from the weight sensor). That is, the control element 314 remains open to continue to impart high flow and the energy source 322 remains on.

However, if the vacuum pressure increases over approximately 1-20 Torr and/or the computer system 310 measures a flowrate of approximately 100-300 g/min within the cannister 306 (based on a reading from the weight sensor), the UIM has been aspirated and the system 300 may move back to the “free flow” state. That is, the control element 314 returns to a partially closed position to impart low flow to prevent blood loss and the laser turns off or not activated. If it is determined that the treatment is complete, the system 300 can be turned off and the user can remove the catheter from the vessel.

Once the UIM has been aspirated, the vacuum pressure increases over approximately 1-20 Torr and/or the computer system 310 measures a flowrate of approximately 100-300 g/min within the cannister 306 (based on a reading from the weight sensor). As a result, the system 300 may move back to the “free flow” state and the energy source 322 may be shut off and/or not activated and the control element 314 may be partially closed to impart low flow. If it is determined that the treatment is complete, the system 300 can be turned off and the user can remove the catheter from the vessel. Table 9 shows a more specific example of sensor measurements that trigger the computer system 310 to move between system states according to either embodiment of 1) the energy source 322 being activated only when there is an obstruction detected or 2) the energy source 322 being activated when there is a UIM detected and when there is an obstruction detected.

TABLE 9

| Sensor input trigger | | |
|---|---|---|
| | Output of weight | |
| Output of vacuum sensor | sensor | Response |
| Stable | Flowrate 15-45 | Remain in previous |
| | g/min | state |
| Increase of over 3 Torr | Flowrate over 300 | If one of two |
| | g/min | conditions occur, |
| | | move to ‘free flow’ |
| Drop of over 5 Torr | — | Move to ‘clot |
| | | detected’ |
| Below 200 Torr | Below 200 g/min | If both conditions |
| | | occur, move to ‘line |
| | | obstructed’ |

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A system for removing an undesirable intravascular material (UIM) from a vasculature of a subject, the system comprising:
a catheter configured to be inserted within the vasculature of the subject;
a canister coupled to the catheter, the canister configured to receive fluid and UIM from the catheter;
a pressure source coupled to the catheter, the pressure source configured to generate a vacuum pressure through the catheter for aspirating the fluid and the UIM;
an energy source coupled to the catheter, the energy source configured to generate energy radiation distally of the catheter to the UIM;
a sensor configured to sense a parameter associated with at least one of the catheter, the canister, or the pressure source; and
a computer system coupled to the sensor, the computer system comprising a processor and a memory, the memory storing instructions that, when executed by the processor, cause the computer system to:
cause the pressure source to initiate the vacuum pressure throughout the catheter,
receive a measurement of the parameter from the sensor,
determine whether the measurement violates a threshold associated with the parameter, and
modulate an aspiration flow rate and/or the energy radiation at a tip of the catheter in response to a determination that the measurement violates the threshold.

2. The system of claim 1, wherein the vacuum pressure is initiated to generate a first aspiration flow rate and the modulated aspiration flow rate is at a second aspiration flow rate, greater than the first aspiration flow rate.

3. The system of claim 1, wherein the modulating of the aspiration flow rate and/or energy radiation includes:
modulating the aspiration flow rate at the tip of the catheter;
receiving another measurement of the parameter from the sensor;
determining whether the another measurement violates the threshold associated with the parameter; and
modulating the energy radiation at the tip of the catheter in response to a determination that the another measurement violates the threshold.

4. The system of claim 3, wherein the causing of the pressure source to initiate the vacuum pressure throughout the catheter includes initiating the vacuum pressure while the energy source is not activated.

5. The system of claim 4, wherein the modulating of the energy radiation includes activating the energy source to generate energy radiation distally of the catheter to the UIM.

6. The system of claim 1, wherein the determination that the measurement violates the threshold corresponds to the tip of the catheter being proximate the UIM in the vasculature or a clog present in the system.

7. The system of claim 1, wherein:
the threshold comprises a target aspiration flow rate; and
modulation of the aspiration flow rate and/or energy radiation causes an aspiration flow rate of the fluid and the UIM to align with the target aspiration flow rate.

8. The system of claim 1, wherein the sensor is selected from the group consisting of a pressure sensor configured to sense the vacuum pressure, a weight sensor configured to sense a weight of the canister, a flow sensor configured to sense an amount of the aspiration, and a current sensor configured to sense a current drawn by the pressure source.

9. A computer-implemented method for removing undesirable intravascular material (UIM) from a subject using a system, the system comprising a catheter configured to be inserted within a vasculature of the subject, a canister coupled to the catheter, the canister configured to receive fluid and the UIM from the catheter, a pressure source coupled to the catheter, the pressure source configured to generate a vacuum pressure through the catheter for aspirating the fluid and the UIM, an energy source coupled to the catheter, the energy source configured to generate energy radiation distally of the catheter to the UIM; and a sensor configured to sense a parameter associated with at least one of the catheter, the canister, or the pressure source, the method comprising:
causing, by a computer system coupled to the pressure source, the energy source and the sensor, the pressure source to initiate the vacuum pressure throughout the catheter;
receiving, by the computer system, a measurement of the parameter from the sensor,
determining, by the computer system, whether the measurement violates a threshold associated with the parameter; and
modulating, by the computer system, an aspiration flow rate and/or the energy source at a tip of the catheter in response to a determination that the measurement violates the threshold.

10. The method of claim 9, wherein the vacuum pressure is initiated to generate a first aspiration flow rate and the modulated aspiration flow rate is at a second aspiration flow rate, greater than the first aspiration flow rate.

11. The method of claim 9, wherein the modulating of the aspiration flow rate and/or energy radiation includes:
modulating the aspiration flow rate at the tip of the catheter;
receiving another measurement of the parameter from the sensor;
determining whether the another measurement violate the threshold associated with the parameter; and
modulating the energy radiation at the tip of the catheter in response to a determination that the another measurement violates the threshold.

12. The method of claim 9, wherein the causing of the pressure source to initiate the vacuum pressure throughout the catheter includes initiating the vacuum pressure while the energy source is not activated.

13. A method comprising:
activating a pump to initiate a first aspiration flow rate to be generated through the catheter to aspirate a fluid and UIM;
receiving a first measurement of a parameter from the sensor; and
determining whether the first measurement violates a threshold associated with the parameter thereby determining a state of the system;
wherein, when the first measurement does not violate the threshold, the first aspiration flow rate is maintained through the catheter,
wherein, when the first measurement is determined to violate the threshold according to a first condition, a second aspiration flow rate is generated through the catheter, wherein the second aspiration flow rate is a faster aspiration flow rate than the first aspiration flow rate; and
wherein, when the first measurement is determined to violate the threshold according to a second condition, the energy source is activated to generate an energy radiation at the distal end of the catheter.

14. The method of claim 13, wherein, when the first measurement does not violate the threshold, the state of the system is determined to be in a free flow state.

15. The method of claim 13, wherein, when the measurement is determined to violate the threshold according to the first condition, the state of the system is determined to be in a UIM detected state.

16. The method of claim 13, wherein, when the measurement is determined to violate the threshold according to the second condition, the state of the system is determined to be in obstructed state.

17. The method of claim 13, wherein, when the measurement is determined to violate the threshold according to the second condition, the second aspiration flow rate is generated through the catheter while the energy source is activated.

18. The method of claim 17, further comprising:
receiving a second measurement of the parameter from the sensor; and
determining whether the second measurement violates the threshold associated with the parameter;
wherein, when the second measurement does not violate the threshold, the energy source remains activated and the second aspiration flow rate is maintained through the catheter, and
wherein, when the second measurement is determined to violate the threshold according to a third condition, the energy source is deactivated and the aspiration flow rate is returned to the first aspiration flow rate through the catheter.

19. The method of claim 18, further comprising:
modulating a control element to cause the aspiration flow rate within the catheter to be returned to the first aspiration flow rate.

20. The method of claim 13, further comprising:
modulating a control element to generate the second aspiration flow rate, wherein the control element is selected from the group consisting of: a valve configured to control the aspiration through tubing connecting the canister to the catheter, an air leak control element configured to modulate the vacuum pressure, a secondary pump configured to control the aspiration through the tubing, and a booster reservoir configured to modulate the vacuum pressure.
